SEQUENCE LISTING (120 sequences)

SEQ ID NO: 1 - DNA, 2732 bp, Homo sapiens
ctttcgcctcagtctcgagctctcgctggccttcgggtgtacgtgctccgggatcttcagcacccgcggccgccatcgccgtcgcttggcttcttctggactcatctgcgccacttgtccgcttcacactccgccgccatcatggtgaagctcgcgaaggcaggtaaaaatcaaggtgaccccaagaaaatggctcctcctccaaaggaggtagaagaagatagtgaagatgaggaaatgtcagaagatgaagaagatgatagcagtggagaagaggtcgtcatacctcagaagaaaggcaagaaggctgctgcaacctcagcaaagaaggtggtcgtttccccaacaaaaaaggttgcagttgccacaccagccaagaaagcagctgtcactccaggcaaaaaggcagcagcaacacctgccaagaagacagttacaccagccaaagcagttaccacacctggcaagaagggagccacaccaggcaaagcattggtagcaactcctggtaagaagggtgctgccatcccagccaagggggcaaagaatggcaagaatgccaagaaggaagacagtgatgaagaggaggatgatgacagtgaggaggatgaggaggatgacgaggacgaggatgaggatgaagatgaaattgaaccagcagcgatgaaagcagcagctgctgcccctgcctcagaggatgaggacgatgaggatgacgaagatgatgaggatgacgatgacgatgaggaagatgactctgaagaagaagctatggagactacaccagccaaaggaaagaaagctgcaaaagttgttcctgtgaaagccaagaacgtggctgaggatgaagatgaagaagaggatgatgaggacgaggatgacgacgacgacgaagatgatgaagatgatgatgatgaagatgatgaggaggaggaagaagaggaggaggaagagcctgtcaaagaagcacctggaaaacgaaagaaggaaatggccaaacagaaagcagctcctgaagccaagaaacagaaagtggaaggcacagaaccgactacggctttcaatctctttgttggaaacctaaactttaacaaatctgctcctgaattaaaaactggtatcagcgatgtttttgctaaaaatgatcttgctgttgtggatgtcagaattggtatgactaggaaatttggttatgtggattttgaatctgctgaagacctggagaaagcgttggaactcactggtttgaaagtctttggcaatgaaattaaactagagaaaccaaaaggaaaagacagtaagaaagagcgagatgcgagaacacttttggctaaaaatctcccttacaaagtcactcaggatgaattgaaagaagtgtttgaagatgctgcggagatcagattagtcagcaaggatgggaaaagtaaagggattgcttatattgaatttaagacagaagctgatgcagagaaaacctttgaagaaaagcagggaacagagatcgatgggcgatctatttccctgtactatactggagagaaaggtcaaaatcaagactatagaggtggaaagaatagcacttggagtggtgaatcaaaaactctggttttaagcaacctctcctacagtgcaacagaagaaactcttcaggaagtatttgagaaagcaacttttatcaaagtaccccagaaccaaaatggcaaatctaaagggtatgcatttatagagtttgcttcattcgaagacgctaaagaagctttaaattcctgtaataaaagggaaattgagggcagagcaatcaggctggagttgcaaggacccaggggatcacctaatgccagaagccagccatccaaaactctgtttgtcaaaggcctgtctgaggataccactgaagagacattaaaggagtcatttgacggctccgttcgggcaaggatagttactgaccgggaaactgggtcctccaaagggtttggttttgtagacttcaacagtgaggaggatgccaaagctgccaaggaggccatggaagacggtgaaattgatggaaataaagttaccttggactgggccaaacctaagggtgaaggtggcttcgggggtcgtggtggaggcagaggcggctttggaggacgaggtggtggtagaggaggccgaggaggatttggtggcagaggccggggaggctttggagggcgaggaggcttccgaggaggcagaggaggaggaggtgaccacaagccacaaggaaagaagacgaagtttgaatagcttctgtccctctgctttcccttttccatttgaaagaaaggactctggggtttttactgttacctgatcaatgacagagccttctgaggacattccaagacagtatacagtcctgtggtctccttggaaatccgtctagttaacatttcaagggcaataccgtgttggttttgactggatattcatataaactttttaaagagttgagtgatagagctaacccttatctgtaagttttgaatttatattgtttcatcccatgtacaaaaccattttttcctacaaatagtttgggttttgttgttgtttcttttttttgttttgtttttgttttttttttttttgcgttcgtggggttgtaaaagaaaagaaagcagaatgttttatcatggtttttgcttcagcggctttaggacaaattaaaagtcaactctggtgccagaaaaaaaaaaaaaaaa

SEQ ID NO: 2 - DNA, 1719 bp, Homo sapiens
gctccacctcgtccgtggccctgcccacccaggccgcaagagctgccgggacggtccccatcttcttggagcgctttaggctggccggcggcgctgggaggtggagtcgttgctgttgctgtttgtgagcctgtggcgcggcttctgtgggccggaaccttaaagatagccgcaatggctgaaaatggtgataatgaaaagatggctgccctggaggccaaaatctgtcatcaaattgagtattattttggcgacttcaatttgccacgggacaagtttctaaaggaacagataaaactggatgaaggctgggtacctttggagataatgataaaattcaacaggttgaaccgtctaacaacagactttaatgtaattgtggaagcattgagcaaatccaaggcagaactcatggaaatcagtgaagataaaactaaaatcagaaggtctccaagcaaacccctacctgaagtgactgatgagtataaaaatgatgtaaaaaacagatctgtttatattaaaggcttcccaactgatgcaactcttgatgacataaaagaatggttagaagataaaggtcaagtactaaatattcagatgagaagaacattgcataaagcatttaagggatcaatttttgttgtgtttgatagcattgaatctgctaagaaatttgtagagacccctggccagaagtacaaagaaacagacctgctaatacttttcaaggacgattactttgccaaaaaaaatgaagaaagaaaacaaaataaagtggaagctaaattaagagctaaacaggagcaagaagcaaaacaaaagttagaagaagatgctgaaatgaaatctctagaagaaaagattggatgcttgctgaaattttcgggtgatttagatgatcagacctgtagagaagatttacacatacttttctcaaatcatggtgaaataaaatggatagacttcgtcagaggagcaaaagaggggataattctatttaaagaaaaagccaaggaagcattgggtaaagccaaagatgcaaataatggtaacctacaattaaggaacaaagaagtgacttgggaagtactagaaggagaggtggaaaaagaagcactgaagaaaataatagaagaccaacaagaatccctaaacaaatggaagtcaaaaggtcgtagatttaaaggaaaaggaaagggtaataaagctgcccagcctgggtctggtaaaggaaaagtacagtttcagggcaagaaaacgaaatttgctagtgatgatgaacatgatgaacatgatgaaaatggtgcaactggacctgtgaaaagagcaagagaagaaacagacaaagaagaacctgcatccaaacaacagaaaacagaaaatggtgctggagaccagtagtttagtaaaccaattttttattcattttaaataggttttaaacgacttttgtttgcggggcttttaaaaggaaaaccgaattaggtccacttcaatgtccacctgtgagaaaggaaaaatttttttgttgtttaacttgtctttttgttatgcaaatgagatttctttgaatgtattgttctgtttgtgttatttcagatgattcaaatatcaaaaggaagattcttccattaaattgcctttgtaatatgagaatgtattagtacaaactaactaataaaatatatactatatgaaaagagcaaaaaaaaaaaaaaa

SEQ ID NO: 3 - DNA, 20 bp, Artificial sequence
  Synthetic oligonucleotide
cggagcacgtacacccgaag

SEQ ID NO: 4 - DNA, 20 bp, Artificial sequence
  Synthetic oligonucleotide
tggcggccgcgggtgctgaa

SEQ ID NO: 5 - DNA, 20 bp, Artificial sequence
  Synthetic oligonucleotide
agatgagtccagaagaagcc

SEQ ID NO: 6 - DNA, 20 bp, Artificial sequence
  Synthetic oligonucleotide
tgaagcggacaagtggcgca

SEQ ID NO: 7 - DNA, 20 bp, Artificial sequence
  Synthetic oligonucleotide
ccatgatggcggcggagtgt

SEQ ID NO: 8 - DNA, 20 bp, Artificial sequence
  Synthetic oligonucleotide
tcctttggaggaggagccat

SEQ ID NO: 9 - DNA, 20 bp, Artificial sequence
  Synthetic oligonucleotide
cctcatcttcactatcttct

SEQ ID NO: 10 - DNA, 20 bp, Artificial sequence
  Synthetic oligonucleotide
tcttcttcatcttctgacat

SEQ ID NO: 11 - DNA, 20 bp, Artificial sequence
  Synthetic oligonucleotide
gacctcttctccactgctat

SEQ ID NO: 12 - DNA, 20 bp, Artificial sequence
  Synthetic oligonucleotide
tgcctttcttctgaggtatg

SEQ ID NO: 13 - DNA, 20 bp, Artificial sequence
  Synthetic oligonucleotide
gctgaggttgcagcagcctt

SEQ ID NO: 14 - DNA, 20 bp, Artificial sequence
  Synthetic oligonucleotide
ggtgtggcaactgcaacctt

SEQ ID NO: 15 - DNA, 20 bp, Artificial sequence
  Synthetic oligonucleotide
gagtgacagctgctttcttg

SEQ ID NO: 16 - DNA, 20 bp, Artificial sequence
  Synthetic oligonucleotide
ctgtcttcttggcaggtgtt

SEQ ID NO: 17 - DNA, 20 bp, Artificial sequence
  Synthetic oligonucleotide
gtaactgctttggctggtgt

SEQ ID NO: 18 - DNA, 20 bp, Artificial sequence
  Synthetic oligonucleotide
ggctcccttcttgccaggtg

SEQ ID NO: 19 - DNA, 20 bp, Artificial sequence
  Synthetic oligonucleotide
gctaccaatgctttgcctgg

SEQ ID NO: 20 - DNA, 20 bp, Artificial sequence
  Synthetic oligonucleotide
agcacccttcttaccaggag

SEQ ID NO: 21 - DNA, 20 bp, Artificial sequence
  Synthetic oligonucleotide
attcttgccattctttgccc

SEQ ID NO: 22 - DNA, 20 bp, Artificial sequence
  Synthetic oligonucleotide
atcactgtcttccttcttgg

SEQ ID NO: 23 - DNA, 20 bp, Artificial sequence
  Synthetic oligonucleotide
cactgtcatcatcctcctct

SEQ ID NO: 24 - DNA, 20 bp, Artificial sequence
  Synthetic oligonucleotide
ttcatcctcatcctcgtcct

SEQ ID NO: 25 - DNA, 20 bp, Artificial sequence
  Synthetic oligonucleotide
gctgctggttcaatttcatc

SEQ ID NO: 26 - DNA, 20 bp, Artificial sequence
  Synthetic oligonucleotide
gcagcagctgctgctttcat

SEQ ID NO: 27 - DNA, 20 bp, Artificial sequence
  Synthetic oligonucleotide
cttcgtcatcctcatcgtcc

SEQ ID NO: 28 - DNA, 20 bp, Artificial sequence
  Synthetic oligonucleotide
gtcatcgtcatcctcatcat

SEQ ID NO: 29 - DNA, 20 bp, Artificial sequence
  Synthetic oligonucleotide
cttcagagtcatcttcctca

SEQ ID NO: 30 - DNA, 20 bp, Artificial sequence
  Synthetic oligonucleotide
gtgtagtctccatagcttct

SEQ ID NO: 31 - DNA, 20 bp, Artificial sequence
  Synthetic oligonucleotide
gcagctttctttcctttggc

SEQ ID NO: 32 - DNA, 20 bp, Artificial sequence
  Synthetic oligonucleotide
ggctttcacaggaacaactt

SEQ ID NO: 33 - DNA, 20 bp, Artificial sequence
  Synthetic oligonucleotide
tcatcctcagccacgttctt

SEQ ID NO: 34 - DNA, 20 bp, Artificial sequence
  Synthetic oligonucleotide
cgtcgtcgtcatcctcgtcc

SEQ ID NO: 35 - DNA, 20 bp, Artificial sequence
  Synthetic oligonucleotide
catcatcttcatcatcttcg

SEQ ID NO: 36 - DNA, 20 bp, Artificial sequence
  Synthetic oligonucleotide
cctcctcatcatcttcatca

SEQ ID NO: 37 - DNA, 20 bp, Artificial sequence
  Synthetic oligonucleotide
tcttcctcctcctcttcttc

SEQ ID NO: 38 - DNA, 20 bp, Artificial sequence
  Synthetic oligonucleotide
ccaggtgcttctttgacagg

SEQ ID NO: 39 - DNA, 20 bp, Artificial sequence
  Synthetic oligonucleotide
gccatttccttctttcgttt

SEQ ID NO: 40 - DNA, 20 bp, Artificial sequence
  Synthetic oligonucleotide
ttcaggagctgctttctgtt

SEQ ID NO: 41 - DNA, 20 bp, Artificial sequence
  Synthetic oligonucleotide
ccttccactttctgtttctt

SEQ ID NO: 42 - DNA, 20 bp, Artificial sequence
  Synthetic oligonucleotide
gaaagccgtagtcggttctg

SEQ ID NO: 43 - DNA, 20 bp, Artificial sequence
  Synthetic oligonucleotide
ttaggtttccaacaaagaga

SEQ ID NO: 44 - DNA, 20 bp, Artificial sequence
  Synthetic oligonucleotide
tcaggagcagatttgttaaa

SEQ ID NO: 45 - DNA, 20 bp, Artificial sequence
  Synthetic oligonucleotide
ttctgacatccacaacagca

SEQ ID NO: 46 - DNA, 20 bp, Artificial sequence
  Synthetic oligonucleotide
cagattcaaaatccacataa

SEQ ID NO: 47 - DNA, 20 bp, Artificial sequence
  Synthetic oligonucleotide
acgctttctccaggtcttca

SEQ ID NO: 48 - DNA, 20 bp, Artificial sequence
  Synthetic oligonucleotide
actttcaaaccagtgagttc

SEQ ID NO: 49 - DNA, 20 bp, Artificial sequence
  Synthetic oligonucleotide
tagtttaatttcattgccaa

SEQ ID NO: 50 - DNA, 20 bp, Artificial sequence
  Synthetic oligonucleotide
tgtcttttccttttggtttc

SEQ ID NO: 51 - DNA, 20 bp, Artificial sequence
  Synthetic oligonucleotide
tcgcatctcgctctttctta

SEQ ID NO: 52 - DNA, 20 bp, Artificial sequence
  Synthetic oligonucleotide
tgactttgtaagggagattt

SEQ ID NO: 53 - DNA, 20 bp, Artificial sequence
  Synthetic oligonucleotide
acttctttcaattcatcctg

SEQ ID NO: 54 - DNA, 20 bp, Artificial sequence
  Synthetic oligonucleotide
gatctccgcagcatcttcaa

SEQ ID NO: 55 - DNA, 20 bp, Artificial sequence
  Synthetic oligonucleotide
cttttcccatccttgctgac

SEQ ID NO: 56 - DNA, 20 bp, Artificial sequence
  Synthetic oligonucleotide
tttctctgcatcagcttctg

SEQ ID NO: 57 - DNA, 20 bp, Artificial sequence
  Synthetic oligonucleotide
ttccctgcttttcttcaaag

SEQ ID NO: 58 - DNA, 20 bp, Artificial sequence
  Synthetic oligonucleotide
atagatcgcccatcgatctc

SEQ ID NO: 59 - DNA, 20 bp, Artificial sequence
  Synthetic oligonucleotide
ctctccagtatagtacaggg

SEQ ID NO: 60 - DNA, 20 bp, Artificial sequence
  Synthetic oligonucleotide
tagtcttgattttgaccttt

SEQ ID NO: 61 - DNA, 20 bp, Artificial sequence
  Synthetic oligonucleotide
agtgctattctttccacctc

SEQ ID NO: 62 - DNA, 20 bp, Artificial sequence
  Synthetic oligonucleotide
ggttgcttaaaaccagagtt

SEQ ID NO: 63 - DNA, 20 bp, Artificial sequence
  Synthetic oligonucleotide
ttctgttgcactgtaggaga

SEQ ID NO: 64 - DNA, 20 bp, Artificial sequence
  Synthetic oligonucleotide
aaatacttcctgaagagttt

SEQ ID NO: 65 - DNA, 20 bp, Artificial sequence
  Synthetic oligonucleotide
ttgataaaagttgctttctc

SEQ ID NO: 66 - DNA, 20 bp, Artificial sequence
  Synthetic oligonucleotide
cataccctttagatttgcca

SEQ ID NO: 67 - DNA, 20 bp, Artificial sequence
  Synthetic oligonucleotide
aatgaagcaaactctataaa

SEQ ID NO: 68 - DNA, 20 bp, Artificial sequence
  Synthetic oligonucleotide
agcttctttagcgtcttcga

SEQ ID NO: 69 - DNA, 20 bp, Artificial sequence
  Synthetic oligonucleotide
cccttttattacaggaattt

SEQ ID NO: 70 - DNA, 20 bp, Artificial sequence
  Synthetic oligonucleotide
tgattgctctgccctcaatt

SEQ ID NO: 71 - DNA, 20 bp, Artificial sequence
  Synthetic oligonucleotide
tgggtccttgcaactccagc

SEQ ID NO: 72 - DNA, 20 bp, Artificial sequence
  Synthetic oligonucleotide
tctggcattaggtgatcccc

SEQ ID NO: 73 - DNA, 20 bp, Artificial sequence
  Synthetic oligonucleotide
acagagttttggatggctgg

SEQ ID NO: 74 - DNA, 20 bp, Artificial sequence
  Synthetic oligonucleotide
tcctcagacaggcctttgac

SEQ ID NO: 75 - DNA, 20 bp, Artificial sequence
  Synthetic oligonucleotide
ctttaatgtctcttcagtgg

SEQ ID NO: 76 - DNA, 20 bp, Artificial sequence
  Synthetic oligonucleotide
gaacggagccgtcaaatgac

SEQ ID NO: 77 - DNA, 20 bp, Artificial sequence
  Synthetic oligonucleotide
cggtcagtaactatccttgc

SEQ ID NO: 78 - DNA, 20 bp, Artificial sequence
  Synthetic oligonucleotide
cagaagctattcaaacttcg

SEQ ID NO: 79 - DNA, 20 bp, Artificial sequence
  Synthetic oligonucleotide
ttgatcaggtaacagtaaaa

SEQ ID NO: 80 - DNA, 20 bp, Artificial sequence
  Synthetic oligonucleotide
atactgtcttggaatgtcct

SEQ ID NO: 81 - DNA, 20 bp, Artificial sequence
  Synthetic oligonucleotide
gatttccaaggagaccacag

SEQ ID NO: 82 - DNA, 20 bp, Artificial sequence
  Synthetic oligonucleotide
acacggtattgcccttgaaa

SEQ ID NO: 83 - DNA, 20 bp, Artificial sequence
  Synthetic oligonucleotide
acctgttgaattttatcatt

SEQ ID NO: 84 - DNA, 20 bp, Artificial sequence
  Synthetic oligonucleotide
agacggttcaacctgttgaa

SEQ ID NO: 85 - DNA, 20 bp, Artificial sequence
  Synthetic oligonucleotide
gtctgttgttagacggttca

SEQ ID NO: 86 - DNA, 20 bp, Artificial sequence
  Synthetic oligonucleotide
ttacattaaagtctgttgtt

SEQ ID NO: 87 - DNA, 20 bp, Artificial sequence
  Synthetic oligonucleotide
gcttccacaattacattaaa

SEQ ID NO: 88 - DNA, 20 bp, Artificial sequence
  Synthetic oligonucleotide
tttgctcaatgcttccacaa

SEQ ID NO: 89 - DNA, 20 bp, Artificial sequence
  Synthetic oligonucleotide
ctgccttggatttgctcaat

SEQ ID NO: 90 - DNA, 20 bp, Artificial sequence
  Synthetic oligonucleotide
tccatgagttctgccttgga

SEQ ID NO: 91 - DNA, 20 bp, Artificial sequence
  Synthetic oligonucleotide
ttcactgatttccatgagtt

SEQ ID NO: 92 - DNA, 20 bp, Artificial sequence
  Synthetic oligonucleotide
tagttttatcttcactgatt

SEQ ID NO: 93 - DNA, 20 bp, Artificial sequence
  Synthetic oligonucleotide
cttctgattttagttttatc

SEQ ID NO: 94 - DNA, 20 bp, Artificial sequence
  Synthetic oligonucleotide
gcttggagaccttctgattt

SEQ ID NO: 95 - DNA, 20 bp, Artificial sequence
  Synthetic oligonucleotide
gtaggggtttgcttggagac

SEQ ID NO: 96 - DNA, 20 bp, Artificial sequence
  Synthetic oligonucleotide
gtcacttcaggtaggggttt

SEQ ID NO: 97 - DNA, 20 bp, Artificial sequence
  Synthetic oligonucleotide
atactcatcagtcacttcag

SEQ ID NO: 98 - DNA, 20 bp, Artificial sequence
  Synthetic oligonucleotide
cctttaatataaacagatct

SEQ ID NO: 99 - DNA, 20 bp, Artificial sequence
  Synthetic oligonucleotide
agtacttgacctttatcttc

SEQ ID NO: 100 - DNA, 20 bp, Artificial sequence
  Synthetic oligonucleotide
aaattgatcccttaaatgct

SEQ ID NO: 101 - DNA, 20 bp, Artificial sequence
  Synthetic oligonucleotide
ttttggcaaagtaatcgtcc

SEQ ID NO: 102 - DNA, 20 bp, Artificial sequence
  Synthetic oligonucleotide
ctcttaatttagcttccact

SEQ ID NO: 103 - DNA, 20 bp, Artificial sequence
  Synthetic oligonucleotide
tttcagcatcttcttctaac

SEQ ID NO: 104 - DNA, 20 bp, Artificial sequence
  Synthetic oligonucleotide
tgctcttttcacaggtccag

SEQ ID NO: 105 - DNA, 20 bp, Artificial sequence
  Synthetic oligonucleotide
ccagcaccattttctgtttt

SEQ ID NO: 106 - DNA, 20 bp, Artificial sequence
  Synthetic oligonucleotide
tttaaaacctatttaaaatg

SEQ ID NO: 107 - DNA, 20 bp, Artificial sequence
  Synthetic oligonucleotide
cccgcaaacaaaagtcgttt

SEQ ID NO: 108 - DNA, 20 bp, Artificial sequence
  Synthetic oligonucleotide
attgaagtggacctaattcg

SEQ ID NO: 109 - DNA, 20 bp, Artificial sequence
  Synthetic oligonucleotide
ctcatttgcataacaaaaag

SEQ ID NO: 110 - DNA, 20 bp, Artificial sequence
  Synthetic oligonucleotide
attctcatattacaaaggca

SEQ ID NO: 111 - DNA, 16 bp, Artificial sequence
  Synthetic oligonucleotide
tttctccaggtcttca

SEQ ID NO: 112 - DNA, 16 bp, Artificial sequence
  Synthetic oligonucleotide
acgctttctccaggtc

SEQ ID NO: 113 - DNA, 18 bp, Artificial sequence
  Synthetic oligonucleotide
gctttctccaggtcttca

SEQ ID NO: 114 - DNA, 18 bp, Artificial sequence
  Synthetic oligonucleotide
acgctttctccaggtctt

SEQ ID NO: 115 - DNA, 16 bp, Artificial sequence
  Synthetic oligonucleotide
gttgcactgtaggaga

SEQ ID NO: 116 - DNA, 16 bp, Artificial sequence
  Synthetic oligonucleotide
ttctgttgcactgtag

SEQ ID NO: 117 - DNA, 18 bp, Artificial sequence
  Synthetic oligonucleotide
ctgttgcactgtaggaga

SEQ ID NO: 118 - DNA, 18 bp, Artificial sequence
  Synthetic oligonucleotide
ttctgttgcactgtagga

SEQ ID NO: 119 - DNA, 20 bp, Artificial sequence
  Synthetic oligonucleotide
accgtctcaaagtcagagtc

SEQ ID NO: 120 - DNA, 20 bp, Artificial sequence
  Synthetic oligonucleotide
ctgctggtacaccgtctcaa

(19) United States
(12) Patent Application Publication
(10) Pub. No.: US 2021/0355493 A1
(43) Pub. Date: Nov. 18, 2021

(54) OLIGONUCLEOTIDE MEDIATED NO-GO DECAY

(71) Applicant: Ionis Pharmaceuticals, Inc., Carlsbad, CA (US)

(72) Inventors: Xue-hai Liang, Del Mar, CA (US); Joshua Nichols, Carlsbad, CA (US); Stanley T. Crooke, Carlsbad, CA (US)

(73) Assignee: Ionis Pharmaceuticals, Inc., Carlsbad, CA (US)

(21) Appl. No.: 17/282,340

(22) PCT Filed: Oct. 4, 2019
(86) PCT No.: PCT/US2019/054671

§ 371 Date: Apr. 1, 2021

Related U.S. Application Data
(60) Provisional application No. 62/742,261, filed on Oct. 5, 2018.

(51) Int. Cl. C12N 15/113

(52) U.S. Cl. CPC C12N 15/113; C12N 2310/322; C12N 2310/321; C12N 2310/3525; C12N 2310/11; C12N 2310/315; C12N 2320/33; C12N 2310/351; C12N 2310/3233

(57) ABSTRACT

The present disclosure provides oligomeric compounds comprising a modified oligonucleotide that induces no-go decay of a target mRNA. In certain embodiments, the modified oligonucleotide is complementary to a region within the 3′ half of the coding region of the target mRNA.

### SEQUENCE LISTING

The present application is being filed along with a Sequence Listing in electronic format. The Sequence Listing is provided as a file entitled CORE0150WOSEQ_ST25.txt created Oct. 2, 2019 which is 32 kb in size. The information in the electronic format of the sequence listing is incorporated herein by reference in its entirety.

### FIELD

The present disclosure provides oligomeric compounds comprising a modified oligonucleotide that modulate no-go mRNA decay. In certain embodiments, the oligomeric compounds induce degradation of a target mRNA.

### BACKGROUND

The principle behind antisense technology is that an antisense compound hybridizes to a target nucleic acid and modulates the amount, activity, and/or function of the target nucleic acid. In one example, target RNA function is modulated via degradation by RNase H upon hybridization with a DNA-like antisense compound. Another example of modulation of gene expression by target degradation is RNA interference (RNAi). RNAi refers to antisense-mediated gene silencing through a mechanism that utilizes the RNA-induced silencing complex (RISC). Regardless of the specific mechanism, sequence specificity makes antisense compounds attractive as tools for target validation and gene functionalization, as well as therapeutics to selectively modulate the expression of genes involved in the pathogenesis of a disease.

Antisense technology is an effective means for modulating the expression of one or more specific gene products and can therefore prove to be uniquely useful in a number of therapeutic, diagnostic, and research applications. Chemically modified nucleosides may be incorporated into antisense compounds to enhance one or more properties, such as nuclease resistance, pharmacokinetics or affinity for a target nucleic acid. No-go decay (NGD) is an mRNA quality control mechanism by which mRNA is degraded during translation that has stalled or arrested. As translation stalls, multiple ribosomes may stack up and collide, and the mRNA is released from the ribosomes following cleavage by a nuclease.

### SUMMARY

The present disclosure provides oligomeric compounds and methods of using oligomeric compounds that modulate no-go decay, wherein the oligomeric compounds comprise a modified oligonucleotide consisting of 18-30 linked nucleosides, wherein the modified oligonucleotide is complementary to a target mRNA, and wherein the target mRNA is a mature mRNA. In certain embodiments, the modified oligonucleotide is less than 90% complementary to the corresponding pre-mRNA of the target mRNA. In certain embodiments, the modified oligonucleotide is at least 90% complementary to a region within the 3′ half of the coding region of the target mRNA, as measured over the entire length of the modified oligonucleotide. In certain embodiments, the modified oligonucleotide is 100% complementary to a region within the 3′ half of the coding region of the target mRNA, as measured over the entire length of the modified oligonucleotide. In certain embodiments, each nucleoside of the modified oligonucleotide comprises a modified sugar moiety. In certain embodiments, each modified sugar moiety is the same modified sugar moiety. In certain embodiments, oligomeric compounds do not alter splicing of the corresponding pre-mRNA of the target mRNA. In certain embodiments, oligomeric compounds induce degradation of the target mRNA, wherein the degradation of the target mRNA occurs via no-go decay, and wherein the degradation of the target mRNA is dependent on HBS1L or PELO expression or activity. In certain embodiments, the target mRNA does not contain a premature termination codon.

### DETAILED DESCRIPTION

It is to be understood that both the foregoing general description and the following detailed description are exemplary and explanatory only and are not restrictive of the embodiments, as claimed. Herein, the use of the singular includes the plural unless specifically stated otherwise. As used herein, the use of “or” means “and/or” unless stated otherwise. Furthermore, the use of the term “including” as well as other forms, such as “includes” and “included,” is not limiting.

The section headings used herein are for organizational purposes only and are not to be construed as limiting the subject matter described. All documents, or portions of documents, cited in this application, including, but not limited to, patents, patent applications, articles, books, treatises, and GenBank and NCBI reference sequence records are hereby expressly incorporated by reference for the portions of the document discussed herein, as well as in their entirety.

It is understood that the sequence set forth in each SEQ ID NO contained herein is independent of any modification to a sugar moiety, an internucleoside linkage, or a nucleobase. As such, compounds defined by a SEQ ID NO may comprise, independently, one or more modifications to a sugar moiety, an internucleoside linkage, or a nucleobase.

As used herein, “2′-deoxyfuranosyl sugar moiety” or “2′-deoxyfuranosyl sugar” means a furanosyl sugar moiety having two hydrogens at the 2′-position. 2′-deoxyfuranosyl sugar moieties may be unmodified or modified and may be substituted at positions other than the 2′-position or unsubstituted. A β-D-2′-deoxyribosyl sugar moiety or 2′-β-D-deoxyribosyl sugar moiety in the context of an oligonucleotide is an unsubstituted, unmodified 2′-deoxyfuranosyl and is found in naturally occurring deoxyribonucleic acids (DNA).

As used herein, “2′-modified” in reference to a furanosyl sugar moiety or nucleoside comprising a furanosyl sugar moiety means the furanosyl sugar moiety comprises a substituent other than H or OH at the 2′-position of the furanosyl sugar moiety. 2′-modified furanosyl sugar moieties include non-bicyclic and bicyclic sugar moieties and may comprise, but are not required to comprise, additional substituents at other positions of the furanosyl sugar moiety.

As used herein, “2′-substituted” in reference to a furanosyl sugar moiety or nucleoside comprising a furanosyl sugar moiety means the furanosyl sugar moiety or nucleoside comprising the furanosyl sugar moiety comprises a substituent other than H or OH at the 2′-position and is a non-bicyclic furanosyl sugar moiety. 2′-substituted furanosyl sugar moieties do not comprise additional substituents at other positions of the furanosyl sugar moiety other than a nucleobase and/or internucleoside linkage(s) when in the context of an oligonucleotide.

As used herein, “ABCE1” means a ATP Binding Cassette Subfamily E Member 1 protein or a nucleic acid that encodes a ATP Binding Cassette Subfamily E Member 1 protein. As used herein, “administration” or “administering” refers to routes of introducing a compound or composition provided herein to a subject to perform its intended function. Examples of routes of administration that can be used include, but are not limited to, administration by inhalation, subcutaneous injection, intrathecal injection, and oral administration .

As used herein, “administered concomitantly” or “co-administration” means administration of two or more compounds in any manner in which the pharmacological effects of both are manifest in the patient.

Concomitant administration does not require that both compounds be administered in a single pharmaceutical composition, in the same dosage form, by the same route of administration, or at the same time. The effects of both compounds need not manifest themselves at the same time. The effects need only be overlapping for a period of time and need not be coextensive. Concomitant administration or co-administration encompasses administration in parallel, sequentially, separate, or simultaneous administration.

As used herein, “animal” refers to a human or non-human animal, including, but not limited to, mice, rats, rabbits, dogs, cats, pigs, and non-human primates, including, but not limited to, monkeys and chimpanzees.

As used herein, “antisense activity” means any detectable and/or measurable change attributable to the hybridization of an antisense compound to its target nucleic acid. In certain embodiments, antisense activity is a decrease in the amount or expression of a target nucleic acid or protein encoded by such target nucleic acid compared to target nucleic acid levels or target protein levels in the absence of the antisense compound.

As used herein, “antisense compound” means a compound comprising an antisense oligonucleotide and optionally one or more additional features, such as a conjugate group or terminal group.

As used herein, “antisense oligonucleotide” means an oligonucleotide having a nucleobase sequence that is at least partially complementary to a target nucleic acid.

As used herein, “ameliorate” in reference to a treatment means improvement in at least one symptom relative to the same symptom in the absence of the treatment. In certain embodiments, amelioration is the reduction in the severity or frequency of a symptom or the delayed onset or slowing of progression in the severity or frequency of a symptom.

As used herein, “bicyclic nucleoside” or “BNA” means a nucleoside comprising a bicyclic sugar moiety. As used herein, “bicyclic sugar” or “bicyclic sugar moiety” means a modified sugar moiety comprising two rings, wherein the second ring is formed via a bridge connecting two of the atoms in the first ring thereby forming a bicyclic structure. In certain embodiments, the first ring of the bicyclic sugar moiety is a furanosyl moiety, and the bicyclic sugar moiety is a modified furanosyl sugar moiety. In certain embodiments, the bicyclic sugar moiety does not comprise a furanosyl moiety.

As used herein, “cEt” or “constrained ethyl” means a bicyclic sugar moiety, wherein the first ring of the bicyclic sugar moiety is a ribosyl sugar moiety, the second ring of the bicyclic sugar is formed via a bridge connecting the 4′-carbon and the 2′-carbon, the bridge has the formula 4′-CH(CH₃)—O-2′, and the methyl group of the bridge is in the S configuration. A cEt bicyclic sugar moiety is in the (β-D configuration.

As used herein, “coding region” in the context of an RNA means the portion of the RNA that is translated into an amino acid sequence. The coding region of an mRNA excludes the 5′-untranslated region and the 3′-untranslated region.

As used herein, “complementary” in reference to an oligonucleotide or a region of an oligonucleotide means that at least 70% of the nucleobases of the entire oligonucleotide or the region of the oligonucleotide, respectively, and the nucleobases of another nucleic acid or one or more regions thereof are capable of hydrogen bonding with one another when the nucleobase sequence of the oligonucleotide and the other nucleic acid are aligned in opposing directions. Complementary nucleobases are nucleobase pairs that are capable of forming hydrogen bonds with one another. Complementary nucleobase pairs include adenine (A) and thymine (T), adenine (A) and uracil (U), cytosine (C) and guanine (G), 5-methyl cytosine (^{m}C) and guanine (G). Complementary oligonucleotides and/or nucleic acids need not have nucleobase complementarity at each nucleoside. Rather, some mismatches are tolerated. As used herein, “fully complementary” or “100% complementary” in reference to oligonucleotides means that such oligonucleotides are complementary to another oligonucleotide or nucleic acid at each nucleoside of the oligonucleotide.

As used herein, “conjugate group” means a group of atoms that is directly or indirectly attached to an oligonucleotide. Conjugate groups may comprise a conjugate moiety and a conjugate linker that attaches the conjugate moiety to the oligonucleotide. As used herein, “conjugate linker” means a group of atoms comprising at least one bond that connects a conjugate moiety to an oligonucleotide.

As used herein, “conjugate moiety” means a group of atoms that is attached to an oligonucleotide via a conjugate linker.

As used herein, “contiguous” or “adjacent” in the context of an oligonucleotide refers to nucleosides, nucleobases, sugar moieties, or internucleoside linkages that are immediately adjacent to each other independent of the other moieties of the oligonucleotide. For example, “contiguous nucleobases” means nucleobases that are immediately adjacent to each other in a sequence. Moieties that are “directly linked” are immediately adjacent to each other and not separated by any other type of moiety.

As used herein, “degradation” in the context of a nucleic acid or protein means at least one cleavage of a contiguous nucleic acid or polypeptide. In certain embodiments, the at least one cleavage is performed by a nuclease.

As used herein, “double-stranded antisense compound” means an antisense compound comprising two oligomeric compounds that are complementary to each other and form a duplex, and wherein one of the two said oligomeric compounds comprises an antisense oligonucleotide.

As used herein, “effective amount” means the amount of compound sufficient to effectuate a desired physiological outcome in a subject in need of the compound. The effective amount may vary among subjects depending on the health and physical condition of the subject to be treated, the taxonomic group of the subjects to be treated, the formulation of the composition, assessment of the subject's medical condition, and other relevant factors.

As used herein, “efficacy” means the ability to produce a desired effect.

As used herein, “exon-exon junction” means a contiguous portion of an mRNA where two exons of a corresponding pre-mRNA were spliced together. An exon-exon junction includes at least one nucleoside of each of the two respective exons and may include up to the entirety of both of the respective exons.

As used herein, “expression” includes all the functions by which a gene's coded information is converted into structures present and operating in a cell. Such structures include, but are not limited to, the products of transcription and translation. As used herein, “modulation of expression” means any change in amount or activity of a product of transcription or translation of a gene. Such a change may be an increase or a reduction of any amount relative to the expression level prior to the modulation.

As used herein, “gapmer” means an oligonucleotide or a portion of an oligonucleotide having a central region comprising a plurality of nucleosides that support RNase H cleavage positioned between a 5′-region and a 3′-region. Herein, the 3′- and 5′-most nucleosides of the central region each comprise a 2′-deoxyfuranosyl sugar moiety. Herein, the 3′-most nucleoside of the 5′-region comprises a 2′-modified sugar moiety or a sugar surrogate. Herein, the 5′-most nucleoside of the 3′-region comprises a 2′-modified sugar moiety or a sugar surrogate. The “central region” may be referred to as a “gap”; and the “5′-region” and “3′-region” may be referred to as “wings”.

As used herein, “HBS1L” means a HBS1 Like Translational GTPase protein or a nucleic acid that encodes a HBS1 Like Translational GTPase protein.

As used herein, “hybridization” means the pairing or annealing of complementary oligonucleotides and/or nucleic acids. While not limited to a particular mechanism, the most common mechanism of hybridization involves hydrogen bonding, which may be Watson-Crick, Hoogsteen or reversed Hoogsteen hydrogen bonding, between complementary nucleobases.

As used herein, “inhibiting the expression or activity” refers to a reduction or blockade of the expression or activity relative to the expression or activity in an untreated or control sample and does not necessarily indicate a total elimination of expression or activity. Inhibition of the expression or activity of a nucleic acid, such as a target mRNA, includes but is not limited to degradation of the nucleic acid.

As used herein, the terms “internucleoside linkage” means a group or bond that forms a covalent linkage between adjacent nucleosides in an oligonucleotide. As used herein “modified internucleoside linkage” means any internucleoside linkage other than a naturally occurring, phosphodiester internucleoside linkage. “Phosphorothioate linkage” means a modified internucleoside linkage in which one of the non-bridging oxygen atoms of a phosphodiester is replaced with a sulfur atom. Modified internucleoside linkages may or may not contain a phosphorus atom. A “neutral internucleoside linkage” is a modified internucleoside linkage that is mostly or completely uncharged at pH 7.4 and/or has a pKa below 7.4.

As used herein, “abasic nucleoside” means a sugar moiety in an oligonucleotide or oligomeric compound that is not directly connected to a nucleobase. In certain embodiments, an abasic nucleoside is adjacent to one or two nucleosides in an oligonucleotide.

As used herein, “LICA-1” is a conjugate group that is represented by the formula:

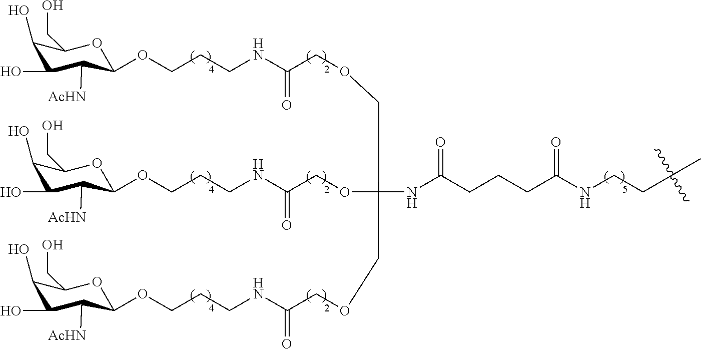

As used herein, “linker-nucleoside” means a nucleoside that links, either directly or indirectly, an oligonucleotide to a conjugate moiety. Linker-nucleosides are located within the conjugate linker of an oligomeric compound. Linker-nucleosides are not considered part of the oligonucleotide portion of an oligomeric compound even if they are contiguous with the oligonucleotide.

As used herein, “non-bicyclic sugar” or “non-bicyclic sugar moiety” means a sugar moiety that comprises fewer than 2 rings. Substituents of modified, non-bicyclic sugar moieties do not form a bridge between two atoms of the sugar moiety to form a second ring.

As used herein, “linked nucleosides” are nucleosides that are connected in a continuous sequence (i.e. no additional nucleosides are present between those that are linked).

As used herein, “mismatch” or “non-complementary” means a nucleobase of a first oligonucleotide that is not complementary with the corresponding nucleobase of a second oligonucleotide or target nucleic acid when the first and second oligomeric compound are aligned.

As used herein, “modulating” refers to changing or adjusting a feature in a cell, tissue, organ or organism.

As used herein, “MOE” means methoxyethyl. “2′-MOE” or “2′-O-methoxyethyl” means a 2′-OCH₂CH₂OCH₃ group at the 2′-position of a furanosyl ring. In certain embodiments, the 2′-OCH₂CH₂OCH₃ group is in place of the 2′-OH group of a ribosyl ring or in place of a 2′-H in a 2′-deoxyribosyl ring.

As used herein, “motif” means the pattern of unmodified and/or modified sugar moieties, nucleobases, and/or internucleoside linkages, in an oligonucleotide or a portion of an oligonucleotide.

As used herein, “naturally occurring” means found in nature.

As used herein, “no-go decay” or “NGD” means a mechanism by which mRNA is degraded during translation, wherein translation is stalled. In certain embodiments, no-go decay requires HBS1L or PELO activity.

As used herein, “nonsense mediated decay” or “NMD” means a mechanism by which mRNA containing a premature termination codon is degraded. In certain embodiments, nonsense mediated decay requires UPF1 or SMG6 activity.

As used herein, “nucleobase” means an unmodified nucleobase or a modified nucleobase. As used herein an “unmodified nucleobase” is adenine (A), thymine (T), cytosine (C), uracil (U), or guanine (G). As used herein, a modified nucleobase is a group of atoms capable of pairing with at least one unmodified nucleobase. A universal base is a nucleobase that can pair with any one of the five unmodified nucleobases. 5-methylcytosine (^{m}C) is one example of a modified nucleobase.

As used herein, “nucleobase sequence” means the order of contiguous nucleobases in a nucleic acid or oligonucleotide independent of any sugar moiety or internucleoside linkage modification.

As used herein, “nucleoside” means a moiety comprising a nucleobase and a sugar moiety. The nucleobase and sugar moiety are each, independently, unmodified or modified. As used herein, “modified nucleoside” means a nucleoside comprising a modified nucleobase and/or a modified sugar moiety.

As used herein, “oligomeric compound” means a compound consisting of an oligonucleotide and optionally one or more additional features, such as a conjugate group or terminal group.

As used herein, “oligonucleotide” means a strand of linked nucleosides connected via internucleoside linkages, wherein each nucleoside and internucleoside linkage may be modified or unmodified. Unless otherwise indicated, oligonucleotides consist of 8-50 linked nucleosides. As used herein, “modified oligonucleotide” means an oligonucleotide, wherein at least one nucleoside or internucleoside linkage is modified. As used herein, “unmodified oligonucleotide” means an oligonucleotide that does not comprise any nucleoside modifications or internucleoside modifications.

As used herein, “PELO” means a Pelota MRNA Surveillance And Ribosome Rescue Factor protein or a nucleic acid that encodes a Pelota MRNA Surveillance And Ribosome Rescue Factor protein.

As used herein, “pharmaceutically acceptable carrier or diluent” means any substance suitable for use in administering to an animal. Certain such carriers enable pharmaceutical compositions to be formulated as, for example, liquids, powders, or suspensions that can be aerosolized or otherwise dispersed for inhalation by a subject. In certain embodiments, a pharmaceutically acceptable carrier or diluent is sterile water; sterile saline; or sterile buffer solution.

As used herein “pharmaceutically acceptable salts” means physiologically and pharmaceutically acceptable salts of compounds, such as oligomeric compounds, i.e., salts that retain the desired biological activity of the compound and do not impart undesired toxicological effects thereto.

As used herein “pharmaceutical composition” means a mixture of substances suitable for administering to a subject. For example, a pharmaceutical composition may comprise an antisense compound and an aqueous solution.

As used herein, “RNAi compound” means an antisense compound that acts, at least in part, through RISC or Ago2 to modulate a target nucleic acid and/or protein encoded by a target nucleic acid. RNAi compounds include, but are not limited to double-stranded siRNA, single-stranded RNA (ssRNA), and microRNA, including microRNA mimics. In certain embodiments, an RNAi compound modulates the amount, activity, and/or splicing of a target nucleic acid. The term RNAi compound excludes antisense oligonucleotides that act through RNase H.

As used herein, the term “single-stranded” in reference to an antisense compound means such a compound consisting of one oligomeric compound that is not paired with a second oligomeric compound to form a duplex. “Self-complementary” in reference to an oligonucleotide means an oligonucleotide that at least partially hybridizes to itself. A compound consisting of one oligomeric compound, wherein the oligonucleotide of the oligomeric compound is self-complementary, is a single-stranded compound. A single-stranded antisense or oligomeric compound may be capable of binding to a complementary oligomeric compound to form a duplex, in which case the compound would no longer be single-stranded.

As used herein, “standard cell assay” means an assay described in any of the Examples, and reasonable variations thereof.

As used herein, “subject” means a human or non-human animal selected for treatment or therapy.

As used herein, “sugar moiety” means an unmodified sugar moiety or a modified sugar moiety. As used herein, “unmodified sugar moiety” means a β-D-ribosyl moiety, as found in naturally occurring RNA, or a β-D-2′-deoxyribosyl sugar moiety as found in naturally occurring DNA. As used herein, “modified sugar moiety” or “modified sugar” means a sugar surrogate or a furanosyl sugar moiety other than a β-D-ribosyl or a β-D-2′-deoxyribosyl. Modified furanosyl sugar moieties may be modified or substituted at a certain position(s) of the sugar moiety, or unsubstituted, and they may or may not have a stereoconfiguration other than β-D-ribosyl. Modified furanosyl sugar moieties include bicyclic sugars and non-bicyclic sugars.

As used herein, “sugar surrogate” means a modified sugar moiety that does not comprise a furanosyl or tetrahydrofuranyl ring (is not a “furanosyl sugar moiety”) and that can link a nucleobase to another group, such as an internucleoside linkage, conjugate group, or terminal group in an oligonucleotide. Modified nucleosides comprising sugar surrogates can be incorporated into one or more positions within an oligonucleotide and such oligonucleotides are capable of hybridizing to complementary oligomeric compounds or nucleic acids.

As used herein, “target” in the context of a nucleic acid, such as an RNA, means a nucleic acid that an oligomeric compound is designed to affect. In certain embodiments, an oligomeric compound comprises an oligonucleotide having a nucleobase sequence that is complementary to more than one RNA, only one of which is the target RNA of the oligomeric compound. In certain embodiments, the target RNA is an RNA present in the species to which an oligomeric compound is administered. In certain embodiments, the target RNA is an mRNA. In certain such embodiments, the target mRNA is a mature mRNA, meaning that the mRNA has already been processed. A mature mRNA excludes a pre-mRNA.

As used herein, “therapeutically effective amount” means an amount of a compound, pharmaceutical agent, or composition that provides a therapeutic benefit to a subject.As used herein, “treat” refers to administering a compound or pharmaceutical composition to an animal in order to effect an alteration or improvement of a disease, disorder, or condition in the animal.

As used herein, a “standard RNase H cleavage assay” is an assay wherein a heteroduplex of the modified oligonucleotide and a complementary strand of unmodified RNA are incubated with each other to form a heteroduplex, and are then incubated with RNase H1 for specified time points before being analyzed on a polyacrylamide gel.

As used herein, a modified nucleoside “supports RNase H cleavage” when incorporated into an oligonucleotide if RNase H cleavage of the complementary RNA is observed within two nucleobases of the modified nucleoside in a standard RNase H cleavage assay.

Certain embodiments are described in the numbered embodiments below:
Embodiment 1. An oligomeric compound comprising a modified oligonucleotide consisting of 18-30 linked nucleosides, wherein the modified oligonucleotide is complementary to a target mRNA, wherein the target mRNA is a mature mRNA, and wherein the modified oligonucleotide is not 100% complementary to a corresponding pre-mRNA of the target mRNA.
Embodiment 2. The oligomeric compound of embodiment 1, wherein the modified oligonucleotide is less than 90% complementary to a corresponding pre-mRNA of the target mRNA.
Embodiment 3. An oligomeric compound comprising a modified oligonucleotide consisting of 18-30 linked nucleosides, wherein the modified oligonucleotide is complementary to a target mRNA, wherein the target mRNA is a mature mRNA, and wherein the modified oligonucleotide is at least 90% complementary to a region within the 3′ half of the coding region of the target mRNA, as measured over the entire length of the modified oligonucleotide.
Embodiment 4. The oligomeric compound of embodiment 3, wherein the modified oligonucleotide is 100% complementary to a region within the 3′ half of the coding region of the target mRNA, as measured over the entire length of the modified oligonucleotide.
Embodiment 5. The oligomeric compound of embodiment 1 or 2, wherein the modified oligonucleotide is at least 90% complementary to a region within the 3′ half of the coding region of the target mRNA, as measured over the entire length of the modified oligonucleotide.
Embodiment 6. The oligomeric compound of embodiment 5, wherein the modified oligonucleotide is 100% complementary to a region within the 3′ half of the coding region of the target mRNA, as measured over the entire length of the modified oligonucleotide.
Embodiment 7. An oligomeric compound comprising a modified oligonucleotide consisting of 18-30 linked nucleosides, wherein the modified oligonucleotide is complementary to a target mRNA, wherein the target mRNA is a mature mRNA, and wherein each nucleoside of the modified oligonucleotide comprises a modified sugar moiety.
Embodiment 8. The oligomeric compound of any of embodiments 1-6, wherein each nucleoside of the modified oligonucleotide comprises a modified sugar moiety.
Embodiment 9. An oligomeric compound comprising a modified oligonucleotide consisting of 18-30 linked nucleosides, wherein the modified oligonucleotide is complementary to a target mRNA, wherein the target mRNA is a mature mRNA, and wherein the oligomeric compound does not alter splicing of a corresponding pre-mRNA of the target mRNA.
Embodiment 10. The oligomeric compound of any of embodiments 1-8, wherein the oligomeric compound does not alter splicing of a corresponding pre-mRNA of the target mRNA.
Embodiment 11. An oligomeric compound comprising a modified oligonucleotide consisting of 18-30 linked nucleosides, wherein the modified oligonucleotide is complementary to a target mRNA, wherein the target mRNA is a mature mRNA, and wherein the oligomeric compound induces degradation of the target mRNA.
Embodiment 12. The oligomeric compound of embodiment 11, wherein the degradation of the target mRNA occurs via no-go decay.
Embodiment 13. The oligomeric compound of embodiment 11 or 12, wherein the degradation of the target mRNA is dependent on HBS1L or PELO expression or activity.
Embodiment 14. The oligomeric compound of any of embodiments 1-10, wherein the oligomeric compound induces degradation of the target mRNA.
Embodiment 15. The oligomeric compound of embodiment 14, wherein the degradation of the target mRNA occurs via no-go decay.
Embodiment 16. The oligomeric compound of embodiment 14 or 15, wherein the degradation of the target mRNA is dependent on HBS1L or PELO expression or activity.
Embodiment 17. An oligomeric compound comprising a modified oligonucleotide consisting of 18-30 linked nucleosides, wherein the modified oligonucleotide is complementary to a target mRNA, wherein the target mRNA is a mature mRNA, and wherein the target mRNA does not contain a premature termination codon.
Embodiment 18. The oligomeric compound of any of embodiments 1-16, wherein the target mRNA does not contain a premature termination codon.
Embodiment 19. The oligomeric compound of any of embodiments 1-18, wherein the modified oligonucleotide consists of 18-24 linked nucleosides.
Embodiment 20. The oligomeric compound of embodiment 19, wherein the modified oligonucleotide consists of 18 linked nucleosides.
Embodiment 21. The oligomeric compound of embodiment 19, wherein the modified oligonucleotide consists of 19 linked nucleosides.
Embodiment 22. The oligomeric compound of embodiment 19, wherein the modified oligonucleotide consists of 20 linked nucleosides.
Embodiment 23. The oligomeric compound of embodiment 19, wherein the modified oligonucleotide consists of 21 linked nucleosides.
Embodiment 24. The oligomeric compound of embodiment 19, wherein the modified oligonucleotide consists of 22 linked nucleosides.
Embodiment 25. The oligomeric compound of embodiment 19, wherein the modified oligonucleotide consists of 23 linked nucleosides.
Embodiment 26. The oligomeric compound of embodiment 19, wherein the modified oligonucleotide consists of 24 linked nucleosides.
Embodiment 27. The oligomeric compound of any of embodiments 1-26, wherein the modified oligonucleotide is not a gapmer.
Embodiment 28. The oligomeric compound of any of embodiments 1-27, wherein the modified oligonucleotide does not comprise 5 or more contiguous nucleosides that each comprise a 2′-deoxyfuranosyl sugar moiety.
Embodiment 29. The oligomeric compound of any of embodiments 1-27, wherein the modified oligonucleotide does not comprise 4 or more contiguous nucleosides that each comprise a 2′-deoxyfuranosyl sugar moiety.
Embodiment 30. The oligomeric compound of any of embodiments 1-29, wherein the modified oligonucleotide does not comprise any 2′-deoxyfuranosyl sugar moieties.
Embodiment 31. The oligomeric compound of any of embodiments 1-30, wherein the modified oligonucleotide comprises at least ten nucleosides each comprising a 2′-substituted furanosyl sugar moiety.
Embodiment 32. The oligomeric compound of any of embodiments 1-30, wherein the modified oligonucleotide comprises at least eleven nucleosides each comprising a 2′-substituted furanosyl sugar moiety.
Embodiment 33. The oligomeric compound of any of embodiments 1-30, wherein the modified oligonucleotide comprises at least twelve nucleosides each comprising a 2′-substituted furanosyl sugar moiety.
Embodiment 34. The oligomeric compound of any of embodiments 1-30, wherein the modified oligonucleotide comprises at least thirteen nucleosides each comprising a 2′-substituted furanosyl sugar moiety.
Embodiment 35. The oligomeric compound of any of embodiments 1-30, wherein the modified oligonucleotide comprises at least fourteen nucleosides each comprising a 2′-substituted furanosyl sugar moiety.
Embodiment 36. The oligomeric compound of any of embodiments 1-30, wherein the modified oligonucleotide comprises at least fifteen nucleosides each comprising a 2′-substituted furanosyl sugar moiety.
Embodiment 37. The oligomeric compound of any of embodiments 1-30, wherein the modified oligonucleotide comprises at least sixteen nucleosides each comprising a 2′-substituted furanosyl sugar moiety.
Embodiment 38. The oligomeric compound of any of embodiments 1-30, wherein the modified oligonucleotide comprises at least seventeen nucleosides each comprising a 2′-substituted furanosyl sugar moiety.
Embodiment 39. The oligomeric compound of any of embodiments 1-30, wherein each nucleoside of the modified oligonucleotide comprises a 2′-substituted furanosyl sugar moiety.
Embodiment 40. The oligomeric compound of embodiment 39, wherein each 2′-substituted furanosyl sugar moiety is the same.
Embodiment 41. The oligomeric compound of any of embodiments 31-40, wherein each 2′-substituted furanosyl sugar moiety is selected from a 2′-O-methyl substituted furanosyl sugar moiety, a 2′-MOE substituted furanosyl sugar moiety, and a 2′-F substituted furanosyl sugar moiety.
Embodiment 42. The oligomeric compound of any of embodiments 31-40, wherein each 2′-substituted sugar moiety is selected from a 2′-O-methyl substituted furanosyl sugar moiety and a 2′-MOE substituted furanosyl sugar moiety.
Embodiment 43. The oligomeric compound of any of embodiments 31-40, wherein each 2′-substituted sugar moiety is a 2′-O-methyl substituted furanosyl sugar moiety.
Embodiment 44. The oligomeric compound of any of embodiments 31-40, wherein each 2′-substituted sugar moiety is a 2′-MOE substituted furanosyl sugar moiety.
Embodiment 45. The oligomeric compound of any of embodiments 1-30, wherein the modified oligonucleotide comprises at least ten nucleosides each comprising a sugar surrogate.
Embodiment 46. The oligomeric compound of any of embodiments 1-30, wherein each nucleoside of the modified oligonucleotide comprises a sugar surrogate.
Embodiment 47. The oligomeric compound of embodiment 46, wherein each sugar surrogate is a morpholino.
Embodiment 48. The oligomeric compound of any of embodiments 11-47, wherein the degradation of the target mRNA is independent of RNase H1 expression or activity.
Embodiment 49. The oligomeric compound of any of embodiments 11-48, wherein the degradation of the target mRNA is independent of nonsense mediated decay.
Embodiment 50. The oligomeric compound of any of embodiments 11-49, wherein the degradation of the target mRNA is independent of UPF1 expression or activity.
Embodiment 51. The oligomeric compound of any of embodiments 11-50, wherein the degradation of the target mRNA is independent of SMG6 expression or activity.
Embodiment 52. The oligomeric compound of any of embodiments 1-51, wherein the oligomeric compound does not bind to RNase H1.
Embodiment 53. The oligomeric compound of any of embodiments 1-52, wherein the oligomeric compound does not support RNase H1 cleavage of the target mRNA.
Embodiment 54. The oligomeric compound of any of embodiments 1-53, wherein the modified oligonucleotide is less than 90% complementary to an exon-exon junction of the target mRNA.
Embodiment 55. The oligomeric compound of any of embodiments 1-53, wherein the modified oligonucleotide is not 100% complementary to an exon-exon junction of the target mRNA.
Embodiment 56. The oligomeric compound of any of embodiments 1-55, wherein the modified oligonucleotide is complementary to a portion of the coding region of the target mRNA that is at least 150 nucleotides downstream from the 5′-end of the coding region of the target mRNA.
Embodiment 57. The oligomeric compound of any of embodiments 1-55, wherein the modified oligonucleotide is complementary to the 3′ most third of the coding region of the target mRNA.
Embodiment 58. The oligomeric compound of any of embodiments 1-55, wherein the modified oligonucleotide is complementary to the 3′ most quarter of the coding region of the target mRNA.
Embodiment 59. The oligomeric compound of any of embodiments 1-58, wherein the modified oligonucleotide is at least 80% complementary to the target mRNA.
Embodiment 60. The oligomeric compound of any of embodiments 1-58, wherein the modified oligonucleotide is at least 85% complementary to the target mRNA.
Embodiment 61. The oligomeric compound of any of embodiments 1-58, wherein the modified oligonucleotide is at least 90% complementary to the target mRNA.
Embodiment 62. The oligomeric compound of any of embodiments 1-58, wherein the modified oligonucleotide is at least 95% complementary to the target mRNA.
Embodiment 63. The oligomeric compound of any of embodiments 1-58, wherein the modified oligonucleotide is 100% complementary to the target mRNA.
Embodiment 64. The oligomeric compound of any of embodiments 1-63, wherein the modified oligonucleotide comprises at lease one modified internucleoside linkage.
Embodiment 65. The oligomeric compound of embodiment 64, wherein the at least one modified internucleoside linkage is a phosphorothioate internucleoside linkage.
Embodiment 66. The oligomeric compound of embodiment 64, wherein each internucleoside linkage of the modified oligonucleotide is a modified internucleoside linkage.
Embodiment 67. The oligomeric compound of embodiment 64 or 66, wherein each modified internucleoside linkage of the modified oligonucleotide is the same modified internucleoside linkage.
Embodiment 68. The oligomeric compound of embodiment 67, wherein each modified internucleoside linkage of the modified oligonucleotide is a phosphorothioate internucleoside linkage.
Embodiment 69. The oligomeric compound of any of embodiments 64-68, wherein each internucleoside linkage of the oligonucleotide is stereorandom.
Embodiment 70. The oligomeric compound of any of embodiments 64-68, wherein at least one internucleoside linkage of the oligonucleotide is chirally controlled.
Embodiment 71. The oligomeric compound of any of embodiments 1-70, wherein the compound comprises a conjugate group.
Embodiment 72. The oligomeric compound of embodiment 71, wherein the conjugate group comprises GalNAc.
Embodiment 73. The oligomeric compound of any of embodiments 1-70, wherein the oligomeric compound consists of the modified oligonucleotide.
Embodiment 74. A method comprising contacting a cell with an oligomeric compound of any of embodiments 1-73.
Embodiment 75. The method of embodiment 74, wherein the target mRNA is degraded.
Embodiment 76. The method of embodiment 75, wherein the target mRNA is degraded by no-go decay.
Embodiment 77. The method of embodiment 74 or 75, wherein the target mRNA degradation is dependent of HBS1L or PELO expression of activity.
Embodiment 78. The method of any of embodiments 74-77, wherein the cell is in an animal.
Embodiment 79. The method of any of embodiments 74-77, wherein the cell is in a human.
Embodiment 80. A method of treating a disease or disorder, comprising administrating the oligomeric compound of any of embodiments 1-73 to an individual in need thereof.
Embodiment 81. The method of embodiment 80, wherein the individual is an animal.
Embodiment 82. The method of embodiment 80, wherein the individual is a human.
Embodiment 83. The method of any of embodiments 80-82, wherein the administration is systemic.
Embodiment 84. The method of embodiment 83, wherein the administration is subcutaneous.
Embodiment 85. The method of any of embodiments 80-82, wherein the administration is intrathecal.
Embodiment 86. The method of any of embodiments 80-82, wherein the administration is via inhalation.
Embodiment 87. The oligomeric compound of any of embodiments 1-73, for use in treating a disease or disorder.

### Certain Compounds

In certain embodiments, compounds described herein are oligomeric compounds comprising or consisting of oligonucleotides consisting of linked nucleosides. Oligonucleotides may be unmodified oligonucleotides or may be modified oligonucleotides. Modified oligonucleotides comprise at least one modification relative to an unmodified oligonucleotide (i.e., comprise at least one modified nucleoside (comprising a modified sugar moiety and/or a modified nucleobase) and/or at least one modified internucleoside linkage).

I. Modifications

A. Modified Nucleosides

Modified nucleosides comprise a modified sugar moiety, a modified nucleobase, or both a modifed sugar moiety and a modified nucleobase.

1. Certain Modified Sugar Moieties

In certain embodiments, sugar moieties are non-bicyclic, modified furanosyl sugar moieties. In certain embodiments, modified sugar moieties are bicyclic or tricyclic furanosyl sugar moieties. In certain embodiments, modified sugar moieties are sugar surrogates. Such sugar surrogates may comprise one or more substitutions corresponding to those of other types of modified sugar moieties.

In certain embodiments, modified sugar moieties are non-bicyclic modified furanosyl sugar moieties comprising one or more acyclic substituent, including but not limited to substituents at the 2′, 4′, and/or 5′ positions. In certain embodiments, the furanosyl sugar moiety is a ribosyl sugar moiety. In certain embodiments one or more acyclic substituent of non-bicyclic modified sugar moieties is branched. Examples of 2′-substituent groups suitable for non-bicyclic modified sugar moieties include but are not limited to: 2′-F, 2′-OCH₃(“OMe” or “O-methyl”), and 2′-O(CH₂)₂OCH₃ (“MOE”). In certain embodiments, 2′-substituent groups are selected from among: halo, allyl, amino, azido, SH, CN, OCN, CF₃, OCF₃, O—C₁-C₁₀ alkoxy, O—C₁-C₁₀ substituted alkoxy, O—C₁-C₁₀ alkyl, O—C₁-C₁₀ substituted alkyl, S-alkyl, N(Rₘ)-alkyl, O-alkenyl, S-alkenyl, N(Rₘ)-alkenyl, O-alkynyl, S-alkynyl, N(Rₘ)-alkynyl, O-alkylenyl-O-alkyl, alkynyl, alkaryl, aralkyl, O-alkaryl, O-aralkyl, O(CH₂)₂SCH₃, O(CH₂)₂ON(Rₘ)(Rₙ) or OCH₂C(═O)—N(Rₘ)(Rₙ), where each Rₘ and Rₙ is, independently, H, an amino protecting group, or substituted or unsubstituted C₁-C₁₀ alkyl, and the 2′-substituent groups described in Cook et al., U.S. Pat. No. 6,531,584; Cook et al., U.S. Pat. No. 5,859,221; and Cook et al., U.S. Pat. No. 6,005,087. Certain embodiments of these 2′-substituent groups can be further substituted with one or more substituent groups independently selected from among: hydroxyl, amino, alkoxy, carboxy, benzyl, phenyl, nitro (NO₂), thiol, thioalkoxy, thioalkyl, halogen, alkyl, aryl, alkenyl and alkynyl. Examples of 4′-substituent groups suitable for non-bicyclic modified sugar moieties include but are not limited to alkoxy (e.g., methoxy), alkyl, and those described in Manoharan et al., WO 2015/106128. Examples of 5′-substituent groups suitable for non-bicyclic modified sugar moieties include but are not limited to: 5′-methyl (R or S), 5′-vinyl, and 5′-methoxy. In certain embodiments, non-bicyclic modified sugars comprise more than one non-bridging sugar substituent, for example, 2′-F-5′-methyl sugar moieties and the modified sugar moieties and modified nucleosides described in Migawa et al., WO 2008/101157 and Rajeev et al., US2013/0203836.

In certain embodiments, a 2′-substituted nucleoside or non-bicyclic 2′-modified nucleoside comprises a sugar moiety comprising a non-bridging 2′-substituent group selected from: F, NH₂, N₃, OCF₃, OCH₃, O(CH₂)₃NH₂, CH₂CH═CH₂, OCH₂CH═CH₂, OCH₂CH₂OCH₃, O(CH₂)₂SCH₃, O(CH₂)₂ON(Rₘ)(Rₙ), O(CH₂)₂O(CH₂)₂N(CH₃)₂, and N-substituted acetamide (OCH₂C(═O)—N(Rₘ)(Rₙ)), where each Rₘ and Rₙ is, independently, H, an amino protecting group, or substituted or unsubstituted C₁-C₁₀ alkyl.

In certain embodiments, a 2′-substituted nucleoside or non-bicyclic 2′-modified nucleoside comprises a sugar moiety comprising a non-bridging 2′-substituent group selected from: F, OCF₃, OCH₃, OCH₂CH₂OCH₃, O(CH₂)₂SCH₃, O(CH₂)₂ON(CH₃)₂, O(CH₂)₂O(CH₂)₂N(CH₃)₂, and OCH₂C(═O)—N(H)CH₃ (“NMA”).

In certain embodiments, a 2′-substituted nucleoside or non-bicyclic 2′-modified nucleoside comprises a sugar moiety comprising a non-bridging 2′-substituent group selected from: F, OCH₃, and OCH₂CH₂OCH₃.

Certain modifed sugar moieties comprise a bridging sugar substituent that forms a second ring resulting in a bicyclic sugar moiety. In certain such embodiments, the bicyclic sugar moiety comprises a bridge between the 4′ and the 2′ furanose ring atoms. In certain such embodiments, the furanose ring is a ribose ring. Examples of sugar moieties comprising such 4′ to 2′ bridging sugar substituents include but are not limited to bicyclic sugars comprising: 4′-CH₂-2′, 4′-(CH₂)₂-2′, 4′-(CH₂)₃-2′, 4′-CH₂—O-2′ (“LNA”), 4′-CH₂-S-2′, 4′-(CH₂)₂—O-2′ (“ENA”), 4′-CH(CH₃)-O-2′ (referred to as “constrained ethyl” or “cEt” when in the S configuration), 4′-CH₂-O—CH₂-2′, 4′-CH₂—N(R)-2′, 4′-CH(CH₂OCH₃)—O-2′ (“constrained MOE” or “cMOE”) and analogs thereof (see, e.g., Seth et al., U.S. Pat. No. 7,399,845, Bhat et al., U.S. Pat. No. 7,569,686, Swayze et al., U.S. Pat. No. 7,741,457, and Swayze et al., U.S. Pat. No. 8,022,193), 4′-C(CH₃)(CH₃)—O-2′ and analogs thereof (see, e.g., Seth et al., U.S. Pat. No. 8,278,283), 4′-CH₂—N(OCH₃)-2′ and analogs thereof (see, e.g., Prakash et al., U.S. Pat. No. 8,278,425), 4′-CH₂—O—N(CH₃)-2′ (see, e.g., Allerson et al., U.S. Pat. No. 7,696,345 and Allerson et al., U.S. Pat. No. 8,124,745), 4′-CH₂—C(H)(CH₃)-2′ (see, e.g., Zhou, et al., J. Org. Chem.,2009, 74, 118-134), 4′-CH₂—C(═CH₂)-2′ and analogs thereof (see e.g., Seth et al., U.S. Pat. No. 8,278,426), 4′-C(RₐR_{b})—N(R)—O-2′, 4′-C(RₐR_{b})-O—N(R)-2′, 4′-CH₂—O—N(R)-2′, and 4′-CH₂—N(R)—O-2′, wherein each R, Rₐ, and R_{b} is, independently, H, a protecting group, or C₁-C₁₂ alkyl (see, e.g. Imanishi et al., U.S. Pat. No. 7,427,672).

In certain embodiments, such 4′ to 2′ bridges independently comprise from 1 to 4 linked groups independently selected from: 4C(Rₐ)(R_{b})ₙ—, —[C(Rₐ)(R_{b})]ₙ—O—, —C(Rₐ)═C(R_{b})—, —C(Rₐ)═N—, —C(═NRₐ)—, —C(═O)—, —C(═S)—, —O—, —S(═O)ₓ—, and —N(Rₐ)—;

wherein:

x is 0, 1, or 2;

n is 1, 2, 3, or 4;

each Rₐ and R_{b}, is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COM, acyl (C(═O)—H), substituted acyl, CN, sulfonyl (S(═O)₂-J₁), or sulfoxyl (S(═O)-J₁); and

each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl, or a protecting group.

Additional bicyclic sugar moieties are known in the art, see, for example: Freier et al., Nucleic Acids Research, 1997, 25(22), 4429-4443, Albaek et al., J. Org. Chem., 2006, 71, 7731-7740, Singh et al., Chem. Commun., 1998, 4, 455-456; Koshkin et al., Tetrahedron, 1998, 54, 3607-3630; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 20017, 129, 8362-8379; Elayadi et al.,; Wengel et a., U.S. Pat. No. 7,053,207; Imanishi et al., U.S. Pat. No. 6,268,490; Imanishi et al. U.S. Pat. No. 6,770,748; Imanishi et al., U.S. RE44,779; Wengel et al., U.S. Pat. No. 6,794,499; Wengel et al., U.S. Pat. No. 6,670,461; Wengel et al., U.S. Pat. No. 7,034,133; Wengel et al., U.S. Pat. No. 8,080,644; Wengel et al., U.S. Pat. No. 8,034,909; Wengel et al., U.S. Pat. No. 8,153,365; Wengel et al., U.S. Pat. No. 7,572,582; and Ramasamy et al., U.S. Pat. No. 6,525,191;; Torsten et al., WO 2004/106356;Wengel et al., WO 1999/014226; Seth et al., WO 2007/134181; Seth et al., U.S. Pat. No. 7,547,684; Seth et al., U.S. Pat. No. 7,666,854; Seth et al., U.S. Pat. No. 8,088,746; Seth et al., U.S. Pat. No. 7,750,131; Seth et al., U.S. Pat. No. 8,030,467; Seth et al., U.S. Pat. No. 8,268,980; Seth et al., U.S. Pat. No. 8,546,556; Seth et al., U.S. Pat. No. 8,530,640; Migawa et al., U.S. Pat. No. 9,012,421; Seth et al., U.S. Pat. No. 8,501,805; and U.S. Patent Publication Nos. Allerson et al., US2008/0039618 and Migawa et al., US2015/0191727.

In certain embodiments, bicyclic sugar moieties and nucleosides incorporating such bicyclic sugar moieties are further defined by isomeric configuration. For example, an LNA nucleoside (described herein) may be in the α-L configuration or in the β-D configuration.

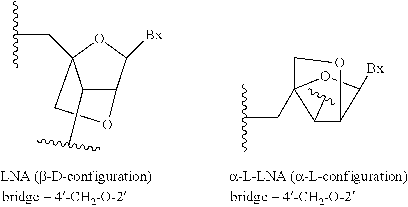

α-L-methyleneoxy (4′-CH₂—O-2′) or α-L-LNA bicyclic nucleosides have been incorporated into antisense oligonucleotides that showed antisense activity (Frieden et al., Nucleic Acids Research, 2003, 21, 6365-6372). Herein, general descriptions of bicyclic nucleosides include both isomeric configurations. When the positions of specific bicyclic nucleosides (e.g., LNA) are identified in exemplified embodiments herein, they are in the β-D configuration, unless otherwise specified.

In certain embodiments, modified sugar moieties comprise one or more non-bridging sugar substituent and one or more bridging sugar substituent (e.g., 5′-substituted and 4′-2′ bridged sugars).

Nucleosides comprising modified furanosyl sugar moieties and modified furanosyl sugar moieties may be referred to by the position(s) of the substitution(s) on the sugar moiety of the nucleoside. The term “modified” following a position of the furanosyl ring, such as “2′-modified”, indicates that the sugar moiety comprises the indicated modification at the 2′ position and may comprise additional modifications and/or substituents. The term “substituted” following a position of the furanosyl ring, such as “2′-substituted” or “2′-4′-substituted”, indicates that is the only position(s) having a substituent other than those found in unmodified sugar moieties in oligonucleotides. Accordingly, the following sugar moieties are represented by the following formulas.

In the context of a nucleoside and/or an oligonucleotide, a non-bicyclic, modified furanosyl sugar moiety is represented by Formula I:

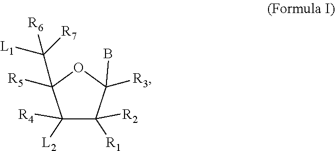

wherein B is a nucleobase; and L₁ and L2 are each, independently, an internucleoside linkage, a terminal group, a conjugate group, or a hydroxyl group. Among the R groups, at least one of R₃₋₇ is not H and/or at least one of R₁ and R₂ is not H or OH. In a 2′-modified furanosyl sugar moiety, at least one of R₁ and R₂ is not H or OH and each of R₃₋₇ is independently selected from H or a substituent other than H. In a 4′-modified furanosyl sugar moiety, R₅ is not H and each of R_{1-4, 6, 7} are independently selected from H and a substituent other than H; and so on for each position of the furanosyl ring. The stereochemistry is not defined unless otherwise noted.

In the context of a nucleoside and/or an oligonucleotide, a non-bicyclic, modified, substituted fuarnosyl sugar moiety is represented by Formula I, wherein B is a nucleobase; and L₁ and L₂ are each, independently, an internucleoside linkage, a terminal group, a conjugate group, or a hydroxyl group. Among the R groups, either one (and no more than one) of R₃₋₇ is a substituent other than H or one of R₁ or R₂ is a substituent other than H or OH. The stereochemistry is not defined unless otherwise noted. Examples of non-bicyclic, modified, substituted furanosyl sugar moieties include 2′-substituted ribosyl, 4′-substituted ribosyl, and 5′-substituted ribosyl sugar moieties, as well as substituted 2′-deoxyfuranosyl sugar moieties, such as 4′-substituted 2′-deoxyribosyl and 5′-substituted 2′-deoxyribosyl sugar moieties.

In the context of a nucleoside and/or an oligonucleotide, a 2′-substituted ribosyl sugar moiety is represented by Formula II:

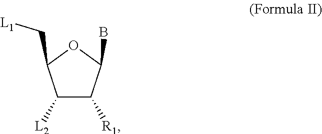

wherein B is a nucleobase; and L₁ and L₂ are each, independently, an internucleoside linkage, a terminal group, a conjugate group, or a hydroxyl group. R₁ is a substituent other than H or OH. The stereochemistry is defined as shown.

In the context of a nucleoside and/or an oligonucleotide, a 4′-substituted ribosyl sugar moiety is represented by Formula III:

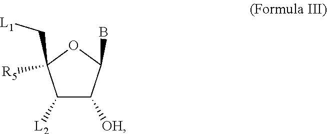

wherein B is a nucleobase; and L₁ and L₂ are each, independently, an internucleoside linkage, a terminal group, a conjugate group, or a hydroxyl group. R₅ is a substituent other than H. The stereochemistry is defined as shown.

In the context of a nucleoside and/or an oligonucleotide, a 5′-substituted ribosyl sugar moiety is represented by Formula IV:

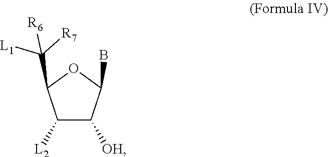

wherein B is a nucleobase; and L₁ and L₂ are each, independently, an internucleoside linkage, a terminal group, a conjugate group, or a hydroxyl group. R₆ or R₇ is a substituent other than H. The stereochemistry is defined as shown.

In the context of a nucleoside and/or an oligonucleotide, a 2′-deoxyfuranosyl sugar moiety is represented by Formula V:

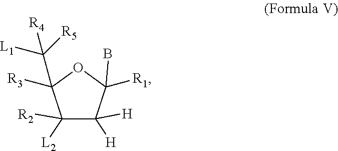

wherein B is a nucleobase; and L₁ and L₂ are each, independently, an internucleoside linkage, a terminal group, a conjugate group, or a hydroxyl group. Each of R₁₋₅ are indepently selected from H and a non-H substituent. If all of R₁₋₅ are each H, the sugar moiety is an unsubstituted 2′-deoxyfuranosyl sugar moiety. The stereochemistry is not defined unless otherwise noted.

In the context of a nucleoside and/or an oligonucleotide, a 4′-substituted 2′-deoxyribosyl sugar moiety is represented by Formula VI:

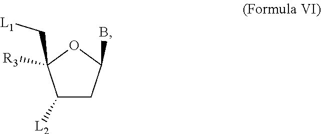

wherein B is a nucleobase; and L₁ and L₂ are each, independently, an internucleoside linkage, a terminal group, a conjugate group, or a hydroxyl group. R₃ is a substituent other than H. The stereochemistry is defined as shown.

In the context of a nucleoside and/or an oligonucleotide, a 5′-substituted 2′-deoxyribosyl sugar moiety is represented by Formula VII:

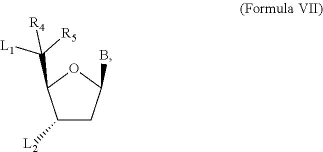

wherein B is a nucleobase; and L₁ and L₂ are each, independently, an internucleoside linkage, a terminal group, a conjugate group, or a hydroxyl group. R₄ or R₅ is a substituent other than H. The stereochemistry is defined as shown.

Unsubstituted 2′-deoxyfuranosyl sugar moieties may be unmodified (β-D-2′-deoxyribosyl) or modified. Examples of modified, unsubstituted 2′-deoxyfuranosyl sugar moieties include β-L-2′-deoxyribosyl, α-L-2′-deoxyribosyl, α-D-2′-deoxyribosyl, and β-D-xylosyl sugar moieties. For example, in the context of a nucleoside and/or an oligonucleotide, a β-L-2′-deoxyribosyl sugar moiety is represented by Formula VIII:

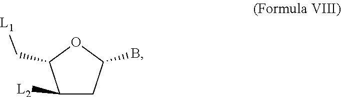

wherein B is a nucleobase; and L₁ and L₂ are each, independently, an internucleoside linkage, a terminal group, a conjugate group, or a hydroxyl group. The stereochemistry is defined as shown.

In certain embodiments, modified sugar moieties are sugar surrogates. In certain such embodiments, the oxygen atom of the sugar moiety is replaced, e.g., with a sulfur, carbon or nitrogen atom. In certain such embodiments, such modified sugar moieties also comprise bridging and/or non-bridging substituents as described herein. For example, certain sugar surrogates comprise a 4′-sulfur atom and a substitution at the 2′-position (see, e.g., Bhat et al., U.S. Pat. No. 7,875,733 and Bhat et al., U.S. Pat. No. 7,939,677) and/or the 5′ position.

In certain embodiments, sugar surrogates comprise rings having other than 5 atoms. For example, in certain embodiments, a sugar surrogate comprises a six-membered tetrahydropyran (“THP”). Such tetrahydropyrans may be further modified or substituted. Nucleosides comprising such modified tetrahydropyrans include but are not limited to hexitol nucleic acid (“HNA”), anitol nucleic acid (“ANA”), manitol nucleic acid (“MNA”) (see, e.g., Leumann, C J. Bioorg. & Med. Chem. 2002, 10, 841-854), fluoro HNA:

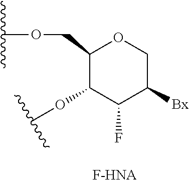

(“F-HNA”, see e.g. Swayze et al., U.S. Pat. No. 8,088,904; Swayze et al., U.S. Pat. No. 8,440,803; Swayze et al., U.S. Pat. No. 8,796,437; and Swayze et al., U.S. Pat. No. 9,005,906; F-HNA can also be referred to as a F-THP or 3′-fluoro tetrahydropyran), and nucleosides comprising additional modified THP compounds represented by Formula IX:

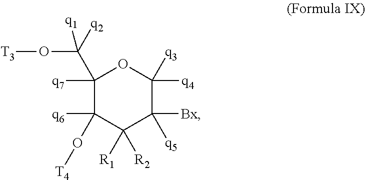

wherein, independently, for each of said modified THP nucleoside:

Bx is a nucleobase moiety;

T₃ and T₄ are each, independently, an internucleoside linkage linking the modified THP nucleoside to the remainder of an oligonucleotide or one of T₃ and T₄ is an internucleoside linkage linking the modified THP nucleoside to the remainder of an oligonucleotide and the other of T3 and T4 is H, a hydroxyl protecting group, a linked conjugate group, or a 5′ or 3′-terminal group; q₁, q₂, q₃, q₄, q₅, q₆ and q₇ are each, independently, H, C₁-C₆ alkyl, substituted C₁-C₆ alkyl, C₂-C6 alkenyl, substituted C₂-C₆ alkenyl, C₂-C₆ alkynyl, or substituted C₂-C₆ alkynyl; and each of R₁ and R₂ is independently selected from among: hydrogen, halogen, substituted or unsubstituted alkoxy, NJ₁J₂, SJ₁, N₃, OC(═X)J₁, OC(═X)NJ₁J₂, NJ₃C(═X)NJ₁J₂, and CN, wherein X is O, S or NJ₁, and each J₁, J₂, and J₃ is, independently, H or C₁-C₆ alkyl.

In certain embodiments, modified THP nucleosides are provided wherein q₁, q₂, q₃, q₄, q₅, q₆ and q₇ are each H. In certain embodiments, at least one of q₁, q₂, q₃, q₄, q₅, q₆ and q₇ is other than H. In certain embodiments, at least one of q₁, q₂, q₃, q₄, q₅, q₆ and q₇ is methyl. In certain embodiments, modified THP nucleosides are provided wherein one of R₁ and R₂ is F. In certain embodiments, R₁ is F and R₂ is H, in certain embodiments, R₁ is methoxy and R₂ is H, and in certain embodiments, R₁ is methoxyethoxy and R₂ is H.

In certain embodiments, sugar surrogates comprise rings having more than 5 atoms and more than one heteroatom. For example, nucleosides comprising morpholino sugar moieties and their use in oligonucleotides have been reported (see, e.g., Braasch et al., Biochemistry, 2002, 41, 4503-4510 and Summerton et al., U.S. Pat. No. 5,698,685; Summerton et al., U.S. Pat. No. 5,166,315; Summerton et al., U.S. Pat. No. 5,185,444; and Summerton et al., U.S. Pat. No. 5,034,506). As used here, the term “morpholino” means a sugar surrogate having the following structure:

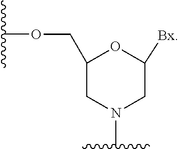

In certain embodiments, morpholinos may be modified, for example by adding or altering various substituent groups from the above morpholino structure. Such sugar surrogates are refered to herein as “modifed morpholinos.”

In certain embodiments, sugar surrogates comprise acyclic moieites. Examples of nucleosides and oligonucleotides comprising such acyclic sugar surrogates include but are not limited to: peptide nucleic acid (“PNA”), acyclic butyl nucleic acid (see, e.g., Kumar et al., Org. Biomol. Chem., 2013, 11, 5853-5865), and nucleosides and oligonucleotides described in Manoharan et al., WO2011/133876.

Many other bicyclic and tricyclic sugar and sugar surrogate ring systems are known in the art that can be used in modified nucleosides.

In certain embodiments, modified nucleosides are DNA mimics. In certain embodiments, a DNA mimic is a sugar surrogate. In certain embodiments, a DNA mimic is a cycohexenyl or hexitol nucleic acid. In certain embodiments, a DNA mimic is described in FIG. 1 of Vester, et. al., “Chemically modified oligonucleotides with efficient RNase H response,” Bioorg. Med. Chem. Letters, 2008, 18: 2296-2300, incorporated by reference herein. In certain embodiments, a DNA mimic nucleoside has a formula selected from:

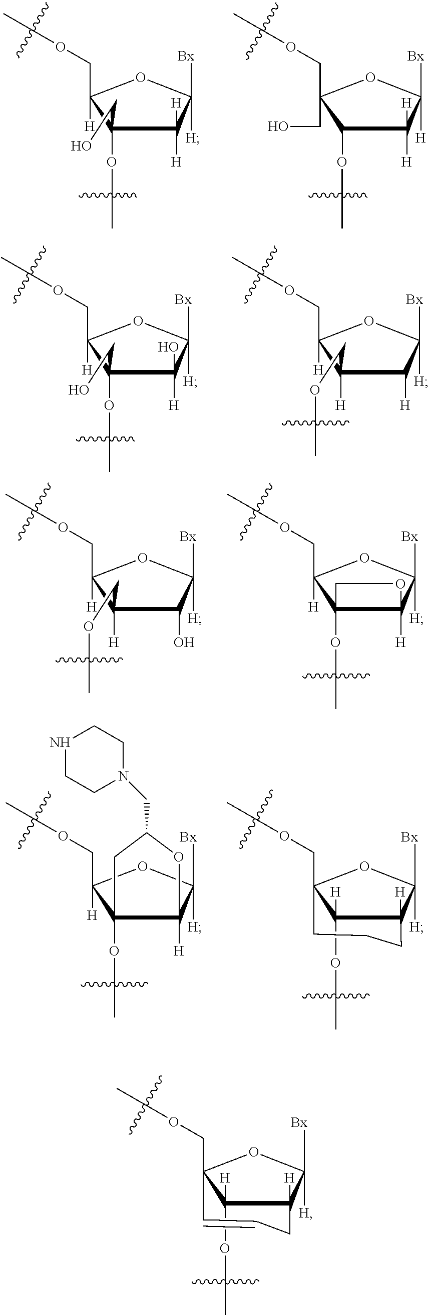

wherein Bx is a heterocyclic base moiety. In certain embodiments, a DNA mimic is α,β-constrained nucleic acid (CAN), 2′,4′-carbocyclic-LNA, or 2′,4′-carbocyclic-ENA. In certain embodiments, a DNA mimic has a sugar moiety selected from among: 4′-C-hydroxymethyl-2′-deoxyribosyl, 3′-C-hydroxymethyl-2′-deoxyribosyl, 3′-C-hydroxymethyl-arabinosyl, 3′-C-2′-O-arabinosyl, 3′-C-methylene-extended-2′-deoxyxylosyl, 3′-C-methylene-extended-xyolosyl, 3′-C-2′-O-piperazino-arabinosyl. In certain embodiments, a DNA mimic has a sugar moiety selected from among: 2′-methylribosyl, 2′-S-methylribosyl, 2′-aminoribosyl, 2′-NH(CH₂)-ribosyl, 2′-NH(CH₂)2-ribosyl, 2′-CH₂-F-ribosyl, 2′-CHF₂-ribosyl, 2′-CF₃-ribosyl, 2′═CF₂ ribosyl, 2′-ethylribosyl, 2′-alkenylribosyl, 2′-alkynylribosyl, 2′-O-4′-C-methyleneribosyl, 2′-cyanoarabinosyl, 2′-chloroarabinosyl, 2′-fluoroarabinosyl, 2′-bromoarabinosyl, 2′-azidoarabinosyl, 2′-methoxyarabinosyl, and 2′-arabinosyl. In certain embodiments, a DNA mimic has a sugar moiety selected from 4′-methyl-modified deoxyfuranosyl, 4′-F-deoxyfuranosyl, 4′-OMe-deoxyfuranosyl. In certain embodiments, a DNA mimic has a sugar moiety selected from among: 5′-methyl-2′-β-D-deoxyribosyl, 5′-ethyl-2′-β-D-deoxyribosyl, 5′-allyl-2′-β-D-deoxyribosyl, 2′-fluoro-β-D-arabinofuranosyl. In certain embodiments, DNA mimics are listed on page 32-33 of PCT/US00/267929 as B-form nucleotides, incorporated by reference herein in its entirety.

2. Modified Nucleobases

In certain embodiments, modified nucleobases are selected from: 5-substituted pyrimidines, 6-azapyrimidines, alkyl or alkynyl substituted pyrimidines, alkyl substituted purines, and N-2, N-6 and O-6 substituted purines. In certain embodiments, modified nucleobases are selected from: 2-aminopropyladenine, 5-hydroxymethyl cytosine, xanthine, hypoxanthine, 2-aminoadenine, 6-N-methylguanine, 6-N-methyladenine, 2-propyladenine , 2-thiouracil, 2-thiothymine and 2-thiocytosine, 5-propynyl (—C≡C—CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in Merigan et al., U.S. Pat. No. 3,687,808, those disclosed in The Concise Encyclopedia Of Polymer Science And Engineering, Kroschwitz, J. I., Ed., John Wiley & Sons, 1990, 858-859; Englisch et al., Angewandte Chemie, International Edition, 1991, 30, 613; Sanghvi, Y. S., Chapter 15, Antisense Research and Applications, Crooke, S. T. and Lebleu, B., Eds., CRC Press, 1993, 273-288; and those disclosed in Chapters 6 and 15, Antisense Drug Technology, Crooke S. T., Ed., CRC Press, 2008, 163-166 and 442-443.

Publications that teach the preparation of certain of the above noted modified nucleobases as well as other modified nucleobases include without limitation, Manoharan et al., US2003/0158403; Manoharan et al., US2003/0175906; Dinh et al., U.S. Pat. No. 4,845,205; Spielvogel et al., U.S. Pat. No. 5,130,302; Rogers et al., U.S. Pat. No. 5,134,066; Bischofberger et al., U.S. Pat. No. 5,175,273; Urdea et al., U.S. Pat. No. 5,367,066; Benner et al., U.S. Pat. No. 5,432,272; Matteucci et al., U.S. Pat. No. 5,434,257; Gmeiner et al., U.S. Pat. No. 5,457,187; Cook et al., U.S. Pat. No. 5,459,255; Froehler et al., U.S. Pat. No. 5,484,908; Matteucci et al., U.S. Pat. No. 5,502,177; Hawkins et al., U.S. Pat. No. 5,525,711; Haralambidis et al., U.S. Pat. No. 5,552,540; Cook et al., U.S. Pat. No. 5,587,469; Froehler et al., U.S. Pat. No. 5,594,121; Switzer et al., U.S. Pat. No. 5,596,091; Cook et al., U.S. Pat. No. 5,614,617; Froehler et al., U.S. Pat. No. 5,645,985; Cook et al., U.S. Pat. No. 5,681,941; Cook et al., U.S. Pat. No. 5,811,534; Cook et al., U.S. Pat. No. 5,750,692; Cook et al., U.S. Pat. No. 5,948,903; Cook et al., U.S. Pat. No. 5,587,470; Cook et al., U.S. Pat. No. 5,457,191; Matteucci et al., U.S. Pat. No. 5,763,588; Froehler et al., U.S. Pat. No. 5,830,653; Cook et al., U.S. Pat. No. 5,808,027; Cook et al., U.S. Pat. No. 6,166,199; and Matteucci et al., U.S. Pat. No. 6,005,096.

In certain embodiments, compounds comprise or consist of a modified oligonucleotide complementary to an target nucleic acid comprising one or more modified nucleobases. In certain embodiments, the modified nucleobase is 5-methylcytosine. In certain embodiments, each cytosine is a 5-methylcytosine.

B. Modified Internucleoside Linkages

In certain embodiments, compounds described herein having one or more modified internucleoside linkages are selected over compounds having only phosphodiester internucleoside linkages because of desirable properties such as, for example, enhanced cellular uptake, enhanced affinity for target nucleic acids, and increased stability in the presence of nucleases.

In certain embodiments, compounds comprise or consist of a modified oligonucleotide complementary to a target nucleic acid comprising one or more modified internucleoside linkages. In certain embodiments, the modified internucleoside linkages are phosphorothioate linkages. In certain embodiments, each internucleoside linkage of an antisense compound is a phosphorothioate internucleoside linkage.

In certain embodiments, nucleosides of modified oligonucleotides may be linked together using any internucleoside linkage. The two main classes of internucleoside linkages are defined by the presence or absence of a phosphorus atom. Representative phosphorus-containing internucleoside linkages include unmodified phosphodiester internucleoside linkages, modified phosphotriesters such as THP phosphotriester and isopropyl phosphotriester, phosphonates such as methylphosphonate, isopropyl phosphonate, isobutyl phosphonate, and phosphonoacetate, phosphoramidates, phosphorothioate, and phosphorodithioate (“HS-P═S”). Representative non-phosphorus containing internucleoside linkages include but are not limited to methylenemethylimino (—CH₂—N(CH₃)—O—CH₂—), thiodiester, thionocarbamate (—O—C(═O)(NH)—S—); siloxane (—O—SiH₂—O—); formacetal, thioacetamido (TANA), alt-thioformacetal, glycine amide, and N,N′-dimethylhydrazine (—CH₂—N(CH₃)—N(CH₃)—). Modified internucleoside linkages, compared to naturally occurring phosphate linkages, can be used to alter, typically increase, nuclease resistance of the oligonucleotide. Methods of preparation of phosphorous-containing and non-phosphorous-containing internucleoside linkages are well known to those skilled in the art.

Representative internucleoside linkages having a chiral center include but are not limited to alkylphosphonates and phosphorothioates. Modified oligonucleotides comprising internucleoside linkages having a chiral center can be prepared as populations of modified oligonucleotides comprising stereorandom internucleoside linkages, or as populations of modified oligonucleotides comprising phosphorothioate linkages in particular stereochemical configurations. In certain embodiments, populations of modified oligonucleotides comprise phosphorothioate internucleoside linkages wherein all of the phosphorothioate internucleoside linkages are stereorandom. Such modified oligonucleotides can be generated using synthetic methods that result in random selection of the stereochemical configuration of each phosphorothioate linkage. Nonetheless, as is well understood by those of skill in the art, each individual phosphorothioate of each individual oligonucleotide molecule has a defined stereoconfiguration. In certain embodiments, populations of modified oligonucleotides are enriched for modified oligonucleotides comprising one or more particular phosphorothioate internucleoside linkages in a particular, independently selected stereochemical configuration. In certain embodiments, the particular configuration of the particular phosphorothioate linkage is present in at least 65% of the molecules in the population. In certain embodiments, the particular configuration of the particular phosphorothioate linkage is present in at least 70% of the molecules in the population. In certain embodiments, the particular configuration of the particular phosphorothioate linkage is present in at least 80% of the molecules in the population. In certain embodiments, the particular configuration of the particular phosphorothioate linkage is present in at least 90% of the molecules in the population. In certain embodiments, the particular configuration of the particular phosphorothioate linkage is present in at least 99% of the molecules in the population. Such chirally enriched populations of modified oligonucleotides can be generated using synthetic methods known in the art, e.g., methods described in Oka et al., JACS 125, 8307 (2003), Wan et al. Nuc. Acid. Res. 42, 13456 (2014), and WO 2017/015555. In certain embodiments, a population of modified oligonucleotides is enriched for modified oligonucleotides having at least one indicated phosphorothioate in the (Sp) configuration. In certain embodiments, a population of modified oligonucleotides is enriched for modified oligonucleotides having at least one phosphorothioate in the (Rp) configuration. In certain embodiments, modified oligonucleotides comprising (Rp) and/or (Sp) phosphorothioates comprise one or more of the following formulas, respectively, wherein “B” indicates a nucleobase:

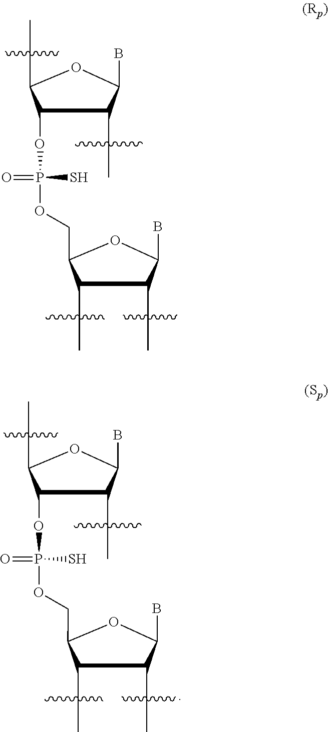

Unless otherwise indicated, chiral internucleoside linkages of modified oligonucleotides described herein can be stereorandom or in a particular stereochemical configuration.

Neutral internucleoside linkages include, without limitation, phosphotriesters, phosphonates, MMI (3′-CH₂—N(CH₃)—O-5′), amide-3 (3′-CH₂—C(═O)—N(H)-5′), amide-4 (3′-CH₂—N(H)—C(═O)-5′), formacetal (3′- O—CH₂-O-5′), methoxypropyl, and thioformacetal (3′-S—CH₂-O-5′). Further neutral internucleoside linkages include nonionic linkages comprising siloxane (dialkylsiloxane), carboxylate ester, carboxamide, sulfide, sulfonate ester and amides (See for example: Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH₂ component parts.

II. Certain Motifs

In certain embodiments, oligomeric compounds described herein comprise or consist of oligonucleotides. Oligonucleotides can have a motif, e.g. a pattern of unmodified and/or modified sugar moieties, nucleobases, and/or internucleoside linkages. In certain embodiments, modified oligonucleotides comprise one or more modified nucleoside comprising a modified sugar. In certain embodiments, modified oligonucleotides comprise one or more modified nucleosides comprising a modified nucleobase. In certain embodiments, modified oligonucleotides comprise one or more modified internucleoside linkage. In such embodiments, the modified, unmodified, and differently modified sugar moieties, nucleobases, and/or internucleoside linkages of a modified oligonucleotide define a pattern or motif. In certain embodiments, the patterns or motifs of sugar moieties, nucleobases, and internucleoside linkages are each independent of one another. Thus, a modified oligonucleotide may be described by its sugar motif, nucleobase motif and/or internucleoside linkage motif (as used herein, nucleobase motif describes the modifications to the nucleobases independent of the sequence of nucleobases).

A. Certain Sugar Motifs

In certain embodiments, oligomeric compounds described herein comprise or consist of oligonucleotides. In certain embodiments, oligonucleotides comprise one or more type of modified sugar and/or unmodified sugar moiety arranged along the oligonucleotide or region thereof in a defined pattern or sugar motif. In certain instances, such sugar motifs include but are not limited to any of the sugar modifications discussed herein.

In certain embodiments, a modified oligonucleotide comprises or has a uniformly modified sugar motif. An oligonucleotide comprising a uniformly modified sugar motif comprises a segment of linked nucleosides, wherein each nucleoside of the segment comprises the same modified sugar moiety. An oligonucleotide having a uniformly modified sugar motif throughout the entirety of the oligonucleotide comprises only nucleosides comprising the same modified sugar moiety. For example, each nucleoside of a 2′-MOE uniformly modified oligonucleotide comprises a 2′-MOE modified sugar moiety. An oligonucleotide comprising or having a uniformly modified sugar motif can have any nucleobase sequence and any internucleoside linkage motif.

B. Certain Nucleobase Motifs

In certain embodiments, oligomeric compounds described herein comprise or consist of oligonucleotides. In certain embodiments, oligonucleotides comprise modified and/or unmodified nucleobases arranged along the oligonucleotide or region thereof in a defined pattern or motif. In certain embodiments, each nucleobase is modified. In certain embodiments, none of the nucleobases are modified. In certain embodiments, each purine or each pyrimidine is modified. In certain embodiments, each adenine is modified. In certain embodiments, each guanine is modified. In certain embodiments, each thymine is modified. In certain embodiments, each uracil is modified. In certain embodiments, each cytosine is modified. In certain embodiments, some or all of the cytosine nucleobases in a modified oligonucleotide are 5-methylcytosines.

In certain embodiments, modified oligonucleotides comprise a block of modified nucleobases. In certain such embodiments, the block is at the 3′-end of the oligonucleotide. In certain embodiments the block is within 3 nucleosides of the 3′-end of the oligonucleotide. In certain embodiments, the block is at the 5′-end of the oligonucleotide. In certain embodiments the block is within 3 nucleosides of the 5′-end of the oligonucleotide.

C. Certain Internucleoside Linkage Motifs

In certain embodiments, oligomeric compounds described herein comprise or consist of oligonucleotides. In certain embodiments, oligonucleotides comprise modified and/or unmodified internucleoside linkages arranged along the oligonucleotide or region thereof in a defined pattern or motif. In certain embodiments, each internucleoside linkage is a phosphodiester internucleoside linkage (P═O). In certain embodiments, each internucleoside linkage of a modified oligonucleotide is a phosphorothioate internucleoside linkage (P═S). In certain embodiments, each internucleoside linkage of a modified oligonucleotide is independently selected from a phosphorothioate internucleoside linkage and phosphodiester internucleoside linkage. In certain embodiments, each phosphorothioate internucleoside linkage is independently selected from a stereorandom phosphorothioate, a (Sp) phosphorothioate, and a (Rp) phosphorothioate. In certain embodiments, the terminal internucleoside linkages are modified. In certain embodiments, the internucleoside linkage motif comprises at least one phosphodiester internucleoside linkage in at least one of the 5′-region and the 3′-region, wherein the at least one phosphodiester linkage is not a terminal internucleoside linkage, and the remaining internucleoside linkages are phosphorothioate internucleoside linkages. In certain such embodiments, all of the phosphorothioate linkages are stereorandom. In certain embodiments, populations of modified oligonucleotides are enriched for modified oligonucleotides comprising such internucleoside linkage motifs.

In certain embodiments, oligonucleotides comprise a region having an alternating internucleoside linkage motif. In certain embodiments, oligonucleotides comprise a region of uniformly modified internucleoside linkages. In certain such embodiments, the internucleoside linkages are phosphorothioate internucleoside linkages. In certain embodiments, all of the internucleoside linkages of the oligonucleotide are phosphorothioate internucleoside linkages. In certain embodiments, each internucleoside linkage of the oligonucleotide is selected from phosphodiester or phosphate and phosphorothioate. In certain embodiments, each internucleoside linkage of the oligonucleotide is selected from phosphodiester or phosphate and phosphorothioate and at least one internucleoside linkage is phosphorothioate.

In certain embodiments, the oligonucleotide comprises at least 6 phosphorothioate internucleoside linkages. In certain embodiments, the oligonucleotide comprises at least 8 phosphorothioate internucleoside linkages. In certain embodiments, the oligonucleotide comprises at least 10 phosphorothioate internucleoside linkages. In certain embodiments, the oligonucleotide comprises at least one block of at least 6 consecutive phosphorothioate internucleoside linkages. In certain embodiments, the oligonucleotide comprises at least one block of at least 8 consecutive phosphorothioate internucleoside linkages. In certain embodiments, the oligonucleotide comprises at least one block of at least 10 consecutive phosphorothioate internucleoside linkages. In certain embodiments, the oligonucleotide comprises at least block of at least one 12 consecutive phosphorothioate internucleoside linkages. In certain such embodiments, at least one such block is located at the 3′ end of the oligonucleotide. In certain such embodiments, at least one such block is located within 3 nucleosides of the 3′ end of the oligonucleotide.

In certain embodiments, oligonucleotides comprise one or more methylphosphonate linkages. In certain embodiments, modified oligonucleotides comprise a linkage motif comprising all phosphorothioate linkages except for one or two methylphosphonate linkages.

In certain embodiments, it is desirable to arrange the number of phosphorothioate internucleoside linkages and phosphodiester internucleoside linkages to maintain nuclease resistance. In certain embodiments, it is desirable to arrange the number and position of phosphorothioate internucleoside linkages and the number and position of phosphodiester internucleoside linkages to maintain nuclease resistance. In certain embodiments, the number of phosphorothioate internucleoside linkages may be decreased and the number of phosphodiester internucleoside linkages may be increased. In certain embodiments, the number of phosphorothioate internucleoside linkages may be decreased and the number of phosphodiester internucleoside linkages may be increased while still maintaining nuclease resistance. In certain embodiments it is desirable to decrease the number of phosphorothioate internucleoside linkages while retaining nuclease resistance. In certain embodiments it is desirable to increase the number of phosphodiester internucleoside linkages while retaining nuclease resistance.

III. Certain Modified Oligonucleotides

In certain embodiments, oligomeric compounds described herein comprise or consist of modified oligonucleotides. In certain embodiments, the above modifications (sugar, nucleobase, internucleoside linkage) are incorporated into a modified oligonucleotide. In certain embodiments, modified oligonucleotides are characterized by their modifications, motifs, and overall lengths. In certain embodiments, such parameters are each independent of one another. Thus, unless otherwise indicated, each internucleoside linkage of a modified oligonucleotide may be modified or unmodified and may or may not follow the modification pattern of the sugar moieties. Likewise, such modified oligonucleotides may comprise one or more modified nucleobase independent of the pattern of the sugar modifications. Furthermore, in certain instances, a modified oligonucleotide is described by an overall length or range and by lengths or length ranges of two or more regions (e.g., a region of nucleosides having specified sugar modifications), in such circumstances it may be possible to select numbers for each range that result in an oligonucleotide having an overall length falling outside the specified range. In such circumstances, both elements must be satisfied. For example, in certain embodiments, a modified oligonucleotide consists of 15-20 linked nucleosides and has a sugar motif consisting of three regions or segments, A, B, and C, wherein region or segment A consists of 2-6 linked nucleosides having a specified sugar motif, region or segment B consists of 6-10 linked nucleosides having a specified sugar motif, and region or segment C consists of 2-6 linked nucleosides having a specified sugar motif. Such embodiments do not include modified oligonucleotides where A and C each consist of 6 linked nucleosides and B consists of 10 linked nucleosides (even though those numbers of nucleosides are permitted within the requirements for A, B, and C) because the overall length of such oligonucleotide is 22, which exceeds the upper limit of 20 for the overall length of the modified oligonucleotide. Unless otherwise indicated, all modifications are independent of nucleobase sequence except that the modified nucleobase 5-methylcytosine is necessarily a “C” in an oligonucleotide sequence.

In certain embodiments, oligonucleotides consist of X to Y linked nucleosides, where X represents the fewest number of nucleosides in the range and Y represents the largest number nucleosides in the range.

In certain such embodiments, X and Y are each independently selected from 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50; provided that X≤Y. For example, in certain embodiments, oligonucleotides consist of 12 to 13, 12 to 14, 12 to 15, 12 to 16, 12 to 17, 12 to 18, 12 to 19, 12 to 20, 12 to 21, 12 to 22, 12 to 23, 12 to 24, 12 to 25, 12 to 26, 12 to 27, 12 to 28, 12 to 29, 12 to 30, 13 to 14, 13 to 15, 13 to 16, 13 to 17, 13 to 18, 13 to 19, 13 to 20, 13 to 21, 13 to 22, 13 to 23, 13 to 24, 13 to 25, 13 to 26, 13 to 27, 13 to 28, 13 to 29, 13 to 30, 14 to 15, 14 to 16, 14 to 17, 14 to 18, 14 to 19, 14 to 20, 14 to 21, 14 to 22, 14 to 23, 14 to 24, 14 to 25, 14 to 26, 14 to 27, 14 to 28, 14 to 29, 14 to 30, 15 to 16, 15 to 17, 15 to 18, 15 to 19, 15 to 20, 15 to 21, 15 to 22, 15 to 23, 15 to 24, 15 to 25, 15 to 26, 15 to 27, 15 to 28, 15 to 29, 15 to 30, 16 to 17, 16 to 18, 16 to 19, 16 to 20, 16 to 21, 16 to 22, 16 to 23, 16 to 24, 16 to 25, 16 to 26, 16 to 27, 16 to 28, 16 to 29, 16 to 30, 17 to 18, 17 to 19, 17 to 20, 17 to 21, 17 to 22, 17 to 23, 17 to 24, 17 to 25, 17 to 26, 17 to 27, 17 to 28, 17 to 29, 17 to 30, 18 to 19, 18 to 20, 18 to 21, 18 to 22, 18 to 23, 18 to 24, 18 to 25, 18 to 26, 18 to 27, 18 to 28, 18 to 29, 18 to 30, 19 to 20, 19 to 21, 19 to 22, 19 to 23, 19 to 24, 19 to 25, 19 to 26, 19 to 29, 19 to 28, 19 to 29, 19 to 30, 20 to 21, 20 to 22, 20 to 23, 20 to 24, 20 to 25, 20 to 26, 20 to 27, 20 to 28, 20 to 29, 20 to 30, 21 to 22, 21 to 23, 21 to 24, 21 to 25, 21 to 26, 21 to 27, 21 to 28, 21 to 29, 21 to 30, 22 to 23, 22 to 24, 22 to 25, 22 to 26, 22 to 27, 22 to 28, 22 to 29, 22 to 30, 23 to 24, 23 to 25, 23 to 26, 23 to 27, 23 to 28, 23 to 29, 23 to 30, 24 to 25, 24 to 26, 24 to 27, 24 to 28, 24 to 29, 24 to 30, 25 to 26, 25 to 27, 25 to 28, 25 to 29, 25 to 30, 26 to 27, 26 to 28, 26 to 29, 26 to 30, 27 to 28, 27 to 29, 27 to 30, 28 to 29, 28 to 30, or 29 to 30 linked nucleosides.

In certain embodiments oligonucleotides have a nucleobase sequence that is complementary to a second oligonucleotide or an identified reference nucleic acid, such as a target nucleic acid. In certain embodiments, a region of an oligonucleotide has a nucleobase sequence that is complementary to a second oligonucleotide or an identified reference nucleic acid, such as a target nucleic acid. In certain embodiments, the nucleobase sequence of a region or entire length of an oligonucleotide is at least 70%, at least 80%, at least 90%, at least 95%, or 100% complementary to the second oligonucleotide or nucleic acid, such as a target nucleic acid.

IV. Certain Conjugated Compounds

In certain embodiments, the oligomeric compounds described herein comprise or consist of an oligonucleotide (modified or unmodified) and optionally one or more conjugate groups and/or terminal groups. Conjugate groups consist of one or more conjugate moiety and a conjugate linker that links the conjugate moiety to the oligonucleotide. Conjugate groups may be attached to either or both ends of an oligonucleotide and/or at any internal position. In certain embodiments, conjugate groups are attached to the 2′-position of a nucleoside of a modified oligonucleotide. In certain embodiments, conjugate groups that are attached to either or both ends of an oligonucleotide are terminal groups. In certain such embodiments, conjugate groups or terminal groups are attached at the 3′ and/or 5′-end of oligonucleotides. In certain such embodiments, conjugate groups (or terminal groups) are attached at the 3′-end of oligonucleotides. In certain embodiments, conjugate groups are attached near the 3′-end of oligonucleotides. In certain embodiments, conjugate groups (or terminal groups) are attached at the 5′-end of oligonucleotides. In certain embodiments, conjugate groups are attached near the 5′-end of oligonucleotides.

Examples of terminal groups include but are not limited to conjugate groups, capping groups, phosphate moieties, protecting groups, modified or unmodified nucleosides, and two or more nucleosides that are independently modified or unmodified.

A. Certain Conjugate Groups

In certain embodiments, oligonucleotides are covalently attached to one or more conjugate groups. In certain embodiments, conjugate groups modify one or more properties of the attached oligonucleotide, including but not limited to pharmacodynamics, pharmacokinetics, stability, binding, absorption, tissue distribution, cellular distribution, cellular uptake, charge and clearance. In certain embodiments, conjugate groups impart a new property on the attached oligonucleotide, e.g., fluorophores or reporter groups that enable detection of the oligonucleotide.

Certain conjugate groups and conjugate moieties have been described previously, for example: cholesterol moiety (Letsinger et al., Proc. Natl. Acad. Sci. USA, 1989, 86, 6553-6556), cholic acid (Manoharan et al., Bioorg. Med. Chem. Lett., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Lett., 1993, 3, 2765-2770), a thiocholesterol (Oberhauser et al., Nucl. Acids Res., 1992, 20, 533-538), an aliphatic chain, e.g., do-decan-diol or undecyl residues (Saison-Behmoaras et al., EMBO 1, 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic, a palmityl moiety (Mishra et al., Biochim. Biophys. Acta, 1995, 1264, 229-237), an octadecylamine or hexylamino-carbonyl-oxycholesterol moiety (Crooke et al., J. Pharmacol. Exp. Ther., 1996, i, 923-937), a tocopherol group (Nishina et al., Molecular Therapy Nucleic Acids, 2015, 4, e220; doi:10.1038/mtna.2014.72 and Nishina et al., Molecular Therapy, 2008, 16, 734-740), or a GalNAc cluster (e.g., WO2014/179620).

1. Conjugate Moieties

Conjugate moieties include, without limitation, intercalators, reporter molecules, polyamines, polyamides, peptides, carbohydrates (e.g., GalNAc), vitamin moieties, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins, fluorophores, and dyes.

In certain embodiments, a conjugate moiety comprises an active drug substance, for example, aspirin, warfarin, phenylbutazone, ibuprofen, suprofen, fen-bufen, ketoprofen, (S)-(+)-pranoprofen, carprofen, dansylsarcosine, 2,3,5-triiodobenzoic acid, fingolimod, flufenamic acid, folinic acid, a benzothiadiazide, chlorothiazide, a diazepine, indo-methicin, a barbiturate, a cephalosporin, a sulfa drug, an antidiabetic, an antibacterial or an antibiotic.

2. Conjugate Linkers

Conjugate moieties are attached to oligonucleotides through conjugate linkers. In certain oligomeric compounds, a conjugate linker is a single chemical bond (i.e. conjugate moiety is attached to an oligonucleotide via a conjugate linker through a single bond). In certain embodiments, the conjugate linker comprises a chain structure, such as a hydrocarbyl chain, or an oligomer of repeating units such as ethylene glycol, nucleosides, or amino acid units.

In certain embodiments, a conjugate linker comprises one or more groups selected from alkyl, amino, oxo, amide, disulfide, polyethylene glycol, ether, thioether, and hydroxylamino. In certain such embodiments, the conjugate linker comprises groups selected from alkyl, amino, oxo, amide and ether groups. In certain embodiments, the conjugate linker comprises groups selected from alkyl and amide groups. In certain embodiments, the conjugate linker comprises groups selected from alkyl and ether groups. In certain embodiments, the conjugate linker comprises at least one phosphorus moiety. In certain embodiments, the conjugate linker comprises at least one phosphate group. In certain embodiments, the conjugate linker includes at least one neutral linking group.

In certain embodiments, conjugate linkers, including the conjugate linkers described above, are bifunctional linking moieties, e.g., those known in the art to be useful for attaching conjugate groups to oligomeric compounds, such as the oligonucleotides provided herein. In general, a bifunctional linking moiety comprises at least two functional groups. One of the functional groups is selected to bind to a particular site on an oligomeric compound and the other is selected to bind to a conjugate group. Examples of functional groups used in a bifunctional linking moiety include but are not limited to electrophiles for reacting with nucleophilic groups and nucleophiles for reacting with electrophilic groups. In certain embodiments, bifunctional linking moieties comprise one or more groups selected from amino, hydroxyl, carboxylic acid, thiol, alkyl, alkenyl, and alkynyl.

Examples of conjugate linkers include but are not limited to pyrrolidine, 8-amino-3,6-dioxaoctanoic acid (ADO), succinimidyl 4-(N-maleimidomethyl) cyclohexane-1-carboxylate (SMCC) and 6-aminohexanoic acid (AHEX or AHA). Other conjugate linkers include but are not limited to substituted or unsubstituted C₁-C₁₀ alkyl, substituted or unsubstituted C₂-C₁₀ alkenyl or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

In certain embodiments, conjugate linkers comprise 1-10 linker-nucleosides. In certain embodiments, such linker-nucleosides are modified nucleosides. In certain embodiments such linker-nucleosides comprise a modified sugar moiety. In certain embodiments, linker-nucleosides are unmodified. In certain embodiments, linker-nucleosides comprise an optionally protected heterocyclic base selected from a purine, substituted purine, pyrimidine or substituted pyrimidine. In certain embodiments, a cleavable moiety is a nucleoside selected from uracil, thymine, cytosine, 4-N-benzoylcytosine, 5-methylcytosine, 4-N-benzoyl-5-methylcytosine, adenine, 6-N-benzoyladenine, guanine and 2-N-isobutyrylguanine. It is typically desirable for linker-nucleosides to be cleaved from the oligomeric compound after it reaches a target tissue. Accordingly, linker-nucleosides are typically linked to one another and to the remainder of the oligomeric compound through cleavable bonds. In certain embodiments, such cleavable bonds are phosphodiester bonds.

Herein, linker-nucleosides are not considered to be part of the oligonucleotide. Accordingly, in embodiments in which an oligomeric compound comprises an oligonucleotide consisting of a specified number or range of linked nucleosides and/or a specified percent complementarity to a reference nucleic acid and the oligomeric compound also comprises a conjugate group comprising a conjugate linker comprising linker-nucleosides, those linker-nucleosides are not counted toward the length of the oligonucleotide and are not used in determining the percent complementarity of the oligonucleotide for the reference nucleic acid. For example, an oligomeric compound may comprise (1) a modified oligonucleotide consisting of 8-30 nucleosides and (2) a conjugate group comprising 1-10 linker-nucleosides that are contiguous with the nucleosides of the modified oligonucleotide. The total number of contiguous linked nucleosides in such a compound is more than 30. Alternatively, an oligomeric compound may comprise a modified oligonucleotide consisting of 8-30 nucleosides and no conjugate group. The total number of contiguous linked nucleosides in such a compound is no more than 30. Unless otherwise indicated conjugate linkers comprise no more than 10 linker-nucleosides. In certain embodiments, conjugate linkers comprise no more than 5 linker-nucleosides. In certain embodiments, conjugate linkers comprise no more than 3 linker-nucleosides. In certain embodiments, conjugate linkers comprise no more than 2 linker-nucleosides. In certain embodiments, conjugate linkers comprise no more than 1 linker-nucleoside.

In certain embodiments, it is desirable for a conjugate group to be cleaved from the oligonucleotide. For example, in certain circumstances oligomeric compounds comprising a particular conjugate moiety are better taken up by a particular cell type, but once the compound has been taken up, it is desirable that the conjugate group be cleaved to release the unconjugated oligonucleotide. Thus, certain conjugate may comprise one or more cleavable moieties, typically within the conjugate linker. In certain embodiments, a cleavable moiety is a cleavable bond. In certain embodiments, a cleavable moiety is a group of atoms comprising at least one cleavable bond. In certain embodiments, a cleavable moiety comprises a group of atoms having one, two, three, four, or more than four cleavable bonds. In certain embodiments, a cleavable moiety is selectively cleaved inside a cell or subcellular compartment, such as a lysosome. In certain embodiments, a cleavable moiety is selectively cleaved by endogenous enzymes, such as nucleases.

In certain embodiments, a cleavable bond is selected from among: an amide, an ester, an ether, one or both esters of a phosphodiester, a phosphate ester, a carbamate, or a disulfide. In certain embodiments, a cleavable bond is one or both of the esters of a phosphodiester. In certain embodiments, a cleavable moiety comprises a phosphate or phosphodiester. In certain embodiments, the cleavable moiety is a phosphate linkage between an oligonucleotide and a conjugate moiety or conjugate group.

In certain embodiments, a cleavable moiety comprises or consists of one or more linker-nucleosides. In certain such embodiments, one or more linker-nucleosides are linked to one another and/or to the remainder of the oligomeric compound through cleavable bonds. In certain embodiments, such cleavable bonds are unmodified phosphodiester bonds. In certain embodiments, a cleavable moiety is 2′-deoxy nucleoside that is attached to either the 3′ or 5′-terminal nucleoside of an oligonucleotide by a phosphate internucleoside linkage and covalently attached to the remainder of the conjugate linker or conjugate moiety by a phosphate or phosphorothioate linkage. In certain such embodiments, the cleavable moiety is 2′-deoxyadenosine.

3. Certain Cell-Targeting Conjugate Moieties

In certain embodiments, a conjugate group comprises a cell-targeting conjugate moiety. In certain embodiments, a conjugate group has the general formula:

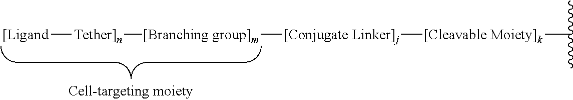

wherein n is from 1 to about 3, m is 0 when n is 1, m is 1 when n is 2 or greater, j is 1 or 0, and k is 1 or 0.

In certain embodiments, n is 1, j is 1 and k is 0. In certain embodiments, n is 1, j is 0 and k is 1. In certain embodiments, n is 1, j is 1 and k is 1. In certain embodiments, n is 2, j is 1 and k is 0. In certain embodiments, n is 2, j is 0 and k is 1. In certain embodiments, n is 2, j is 1 and k is 1. In certain embodiments, n is 3, j is 1 and k is 0. In certain embodiments, n is 3, j is 0 and k is 1. In certain embodiments, n is 3, j is 1 and k is 1.

In certain embodiments, conjugate groups comprise cell-targeting moieties that have at least one tethered ligand. In certain embodiments, cell-targeting moieties comprise two tethered ligands covalently attached to a branching group. In certain embodiments, cell-targeting moieties comprise three tethered ligands covalently attached to a branching group.

In certain embodiments, the cell-targeting moiety comprises a branching group comprising one or more groups selected from alkyl, amino, oxo, amide, disulfide, polyethylene glycol, ether, thioether and hydroxylamino groups. In certain embodiments, the branching group comprises a branched aliphatic group comprising groups selected from alkyl, amino, oxo, amide, disulfide, polyethylene glycol, ether, thioether and hydroxylamino groups. In certain such embodiments, the branched aliphatic group comprises groups selected from alkyl, amino, oxo, amide and ether groups. In certain such embodiments, the branched aliphatic group comprises groups selected from alkyl, amino and ether groups. In certain such embodiments, the branched aliphatic group comprises groups selected from alkyl and ether groups. In certain embodiments, the branching group comprises a mono or polycyclic ring system.

In certain embodiments, each tether of a cell-targeting moiety comprises one or more groups selected from alkyl, substituted alkyl, ether, thioether, disulfide, amino, oxo, amide, phosphodiester, and polyethylene glycol, in any combination. In certain embodiments, each tether is a linear aliphatic group comprising one or more groups selected from alkyl, ether, thioether, disulfide, amino, oxo, amide, and polyethylene glycol, in any combination. In certain embodiments, each tether is a linear aliphatic group comprising one or more groups selected from alkyl, phosphodiester, ether, amino, oxo, and amide, in any combination. In certain embodiments, each tether is a linear aliphatic group comprising one or more groups selected from alkyl, ether, amino, oxo, and amid, in any combination. In certain embodiments, each tether is a linear aliphatic group comprising one or more groups selected from alkyl, amino, and oxo, in any combination. In certain embodiments, each tether is a linear aliphatic group comprising one or more groups selected from alkyl and oxo, in any combination. In certain embodiments, each tether is a linear aliphatic group comprising one or more groups selected from alkyl and phosphodiester, in any combination. In certain embodiments, each tether comprises at least one phosphorus linking group or neutral linking group. In certain embodiments, each tether comprises a chain from about 6 to about 20 atoms in length. In certain embodiments, each tether comprises a chain from about 10 to about 18 atoms in length. In certain embodiments, each tether comprises about 10 atoms in chain length.

In certain embodiments, each ligand of a cell-targeting moiety has an affinity for at least one type of receptor on a target cell. In certain embodiments, each ligand has an affinity for at least one type of receptor on the surface of a mammalian lung cell.

In certain embodiments, each ligand of a cell-targeting moiety is a carbohydrate, carbohydrate derivative, modified carbohydrate, polysaccharide, modified polysaccharide, or polysaccharide derivative. In certain such embodiments, the conjugate group comprises a carbohydrate cluster (see, e.g., Maier et al., “Synthesis of Antisense Oligonucleotides Conjugated to a Multivalent Carbohydrate Cluster for Cellular Targeting,” Bioconjugate Chemistry, 2003, 14, 18-29, or Rensen et al., “Design and Synthesis of Novel N-Acetylgalactosamine-Terminated Glycolipids for Targeting of Lipoproteins to the Hepatic Asiaglycoprotein Receptor,” J. Med. Chem. 2004, 47, 5798-5808, which are incorporated herein by reference in their entirety).

In certain such embodiments, each ligand is an amino sugar or a thio sugar. For example, amino sugars may be selected from any number of compounds known in the art, such as sialic acid, a-D-galactosamine, (3-muramic acid, 2-deoxy-2-methylamino-L-glucopyranose, 4,6-dideoxy-4-formamido-2,3-di-O-methyl-D-mannopyranose, 2-deoxy-2-sulfoamino-D-glucopyranose and N-sulfo-D-glucosamine, and N-glycoloyl-α-neuraminic acid. For example, thio sugars may be selected from 5-Thio-β-D-glucopyranose, methyl 2,3,4-tri-O-acetyl-1-thio-6-O-trityl-α-D-glucopyranoside, 4-thio-β-D-galactopyranose, and ethyl 3,4,6,7-tetra-O-acetyl-2-deoxy-1,5-dithio-α-D-gluco-heptopyranoside.

In certain embodiments, oligomeric compounds described herein comprise a conjugate group found in any of the following references: Lee, Carbohydr Res, 1978, 67, 509-514; Connolly et al., J Biol Chem, 1982, 257, 939-945; Pavia et al., Int J Pep Protein Res, 1983, 22, 539-548; Lee et al., Biochem, 1984, 23, 4255-4261; Lee et al., Glycoconjugate J, 1987, 4, 317-328; Toyokuni et al., Tetrahedron Lett, 1990, 31, 2673-2676; Biessen et al., J Med Chem, 1995, 38, 1538-1546; Valentijn et al., Tetrahedron, 1997, 53, 759-770; Kim et al., Tetrahedron Lett, 1997, 38, 3487-3490; Lee et al., Bioconjug Chem, 1997, 8, 762-765; Kato et al., Glycobiol, 2001, 11, 821-829; Rensen et al., J Biol Chem, 2001, 276, 37577-37584; Lee et al., Methods Enzymol, 2003, 362, 38-43; Westerlind et al., Glycoconj J, 2004, 21, 227-241; Lee et al., Bioorg Med Chem Lett, 2006, 16(19), 5132-5135; Maierhofer et al., Bioorg Med Chem, 2007, 15, 7661-7676; Khorev et al., Bioorg Med Chem, 2008, 16, 5216-5231; Lee et al., Bioorg Med Chem, 2011, 19, 2494-2500; Kornilova et al., Analyt Biochem, 2012, 425, 43-46; Pujol et al., Angew Chemie Int Ed Engl, 2012, 51, 7445-7448; Biessen et al., J Med Chem, 1995, 38, 1846-1852; Sliedregt et al., J Med Chem, 1999, 42, 609-618; Rensen et al., J Med Chem, 2004, 47, 5798-5808; Rensen et al., Arterioscler Thromb Vasc Biol, 2006, 26, 169-175; van Rossenberg et al., Gene Ther, 2004, 11, 457-464; Sato et al., J Am Chem Soc, 2004, 126, 14013-14022; Lee et al., J Org Chem, 2012, 77, 7564-7571; Biessen et al., FASEB J, 2000, 14, 1784-1792; Rajur et al., Bioconjug Chem, 1997, 8, 935-940; Duff et al., Methods Enzymol, 2000, 313, 297-321; Maier et al., Bioconjug Chem, 2003, 14, 18-29; Jayaprakash et al., Org Lett, 2010, 12, 5410-5413; Manoharan, Antisense Nucleic Acid Drug Dev, 2002, 12, 103-128; Merwin et al., Bioconjug Chem, 1994, 5, 612-620; Tomiya et al., Bioorg Med Chem, 2013, 21, 5275-5281; International applications WO1998/013381; WO2011/038356; WO1997/046098; WO2008/098788; WO2004/101619; WO2012/037254; WO2011/120053; WO2011/100131; WO2011/163121; WO2012/177947; WO2013/033230; WO2013/075035; WO2012/083185; WO2012/083046; WO2009/082607; WO2009/134487; WO2010/144740; WO2010/148013; WO1997/020563; WO2010/088537; WO2002/043771; WO2010/129709; WO2012/068187; WO2009/126933; WO2004/024757; WO2010/054406; WO2012/089352; WO2012/089602; WO2013/166121; WO2013/165816; U.S. Pat. Nos. 4,751,219; 8,552,163; 6,908,903; 7,262,177; 5,994,517; 6,300,319; 8,106,022; 7,491,805; 7,491,805; 7,582,744; 8,137,695; 6,383,812; 6,525,031; 6,660,720; 7,723,509; 8,541,548; 8,344,125; 8,313,772; 8,349,308; 8,450,467; 8,501,930; 8,158,601; 7,262,177; 6,906,182; 6,620,916; 8,435,491; 8,404,862; 7,851,615; Published U.S. Patent Application Publications US2011/0097264; US2011/0097265; US2013/0004427; US2005/0164235; US2006/0148740; US2008/0281044; US2010/0240730; US2003/0119724; US2006/0183886; US2008/0206869; US2011/0269814; US2009/0286973; US2011/0207799; US2012/0136042; US2012/0165393; US2008/0281041; US2009/0203135; US2012/0035115; US2012/0095075; US2012/0101148; US2012/0128760; US2012/0157509; US2012/0230938; US2013/0109817; US2013/0121954; US2013/0178512; US2013/0236968; US2011/0123520; US2003/0077829; US2008/0108801; and US2009/0203132.

### Compositions and Methods for Formulating Pharmaceutical Compositions

Oligomeric compounds described herein may be admixed with pharmaceutically acceptable active or inert substances for the preparation of pharmaceutical compositions. Compositions and methods for the formulation of pharmaceutical compositions are dependent upon a number of criteria, including, but not limited to, route of administration, extent of disease, or dose to be administered.

Certain embodiments provide pharmaceutical compositions comprising one or more oligomeric compounds or a salt thereof. In certain embodiments, the oligomeric compounds comprise or consist of a modified oligonucleotide. In certain such embodiments, the pharmaceutical composition comprises a suitable pharmaceutically acceptable diluent or carrier. In certain embodiments, a pharmaceutical composition comprises a sterile saline solution and one or more oligomeric compound. In certain embodiments, such pharmaceutical composition consists of a sterile saline solution and one or more oligomeric compound. In certain embodiments, the sterile saline is pharmaceutical grade saline. In certain embodiments, a pharmaceutical composition comprises one or more oligomeric compound and sterile water. In certain embodiments, a pharmaceutical composition consists of one oligomeric compound and sterile water. In certain embodiments, the sterile water is pharmaceutical grade water. In certain embodiments, a pharmaceutical composition comprises or consists of one or more oligomeric compound and phosphate-buffered saline (PBS). In certain embodiments, a pharmaceutical composition consists of one or more oligomeric compound and sterile PBS. In certain embodiments, the sterile PBS is pharmaceutical grade PBS. Compositions and methods for the formulation of pharmaceutical compositions are dependent upon a number of criteria, including, but not limited to, route of administration, extent of disease, or dose to be administered.

An oligomeric compound described herein complementary to a target nucleic acid can be utilized in pharmaceutical compositions by combining the oligomeric compound with a suitable pharmaceutically acceptable diluent or carrier and/or additional components such that the pharmaceutical composition is suitable for injection. In certain embodiments, a pharmaceutically acceptable diluent is phosphate buffered saline. Accordingly, in one embodiment, employed in the methods described herein is a pharmaceutical composition comprising an oligomeric compound complementary to a target nucleic acid and a pharmaceutically acceptable diluent. In certain embodiments, the pharmaceutically acceptable diluent is phosphate buffered saline. In certain embodiments, the oligomeric compound comprises or consists of a modified oligonucleotide provided herein.

Pharmaceutical compositions comprising oligomeric compounds provided herein encompass any pharmaceutically acceptable salts, esters, or salts of such esters, or any other oligonucleotide which, upon administration to an animal, including a human, is capable of providing (directly or indirectly) the biologically active metabolite or residue thereof. In certain embodiments, the oligomeric compound comprises or consists of a modified oligonucleotide. Accordingly, for example, the disclosure is also drawn to pharmaceutically acceptable salts of compounds, prodrugs, pharmaceutically acceptable salts of such prodrugs, and other bioequivalents. Suitable pharmaceutically acceptable salts include, but are not limited to, sodium and potassium salts.

### Certain Mechanisms

No-go decay is a mechanism that serves to degrade mRNA undergoing translation that has stalled or stopped. The stalled ribosomes are released from the mRNA, and the mRNA is cleaved by a nuclease near the site of the stalled ribosomes, typically near the 3′-end of the mRNA. Nonsense mediated decay is a distinct mechanism that serves to degrade mRNA that contains a premature termination codon. No-go decay in human cells requires HBS1L, PELO, and/or ABCE1 activity.

In certain embodiments, the oligomeric compounds described herein are capable of sterically blocking ribosome progression on the mRNA or blocking elongation of translation and such modulation causes the degradation and/or reduction of the target mRNA through no-go decay. In certain embodiments, oligomeric compounds capable of sterically blocking ribosome progression on an mRNA are complementary to a portion of the 3′ half of the coding region of the mRNA. In certain such embodiments, oligomeric compounds capable of sterically blocking ribosome progression on an mRNA are complementary to a portion of the coding region of the mRNA within 200, 300, 400, 500, 600, 700, or 800 nucleotides of the 3′-end of the coding region.

In certain embodiments, the target mRNA does not contain a premature termination codon and is not subject to nonsense mediated decay. In certain embodiments, the oligomeric compound does not alter splicing of the pre-mRNA that is processed to become the target mRNA (the corresponding pre-mRNA). In such certain embodiments, the oligomeric compound is not 100% complementary to the corresponding pre-mRNA. In certain embodiments, the oligomeric compound is 100% complementary to the corresponding pre-mRNA but does not alter splicing.

In certain embodiments, oligomeric compounds induce degradation of a target mRNA via more than one mechanism. In certain such embodiments, oligomeric compounds herein modulate the amount or activity of a target nucleic acid through no-go decay pathway to a greater extent than they modulate the amount or activity of a target nucleic acid through another mechanism. For example, in certain embodiments, an oligomeric compound modulates the amount or activity of a target nucleic acid through no-go decay to a greater extent than it modulates the amount or activity of a target nucleic acid through through RNase H. The extent of modulation through no-go decay is greater than the extent of modulation through RNase H when, for example, the concentration of oligomeric compound required to modulate the target mRNA in the absence of no-go decay pathway members is much higher than the concentration of oligomeric compound required to modulate the target mRNA in the absence of RNase H.

Antisense activities, such as degradation via no-go decay may be observed directly or indirectly. In certain embodiments, observation or detection of an antisense activity involves observation or detection of a change in an amount of a target nucleic acid or protein encoded by such target nucleic acid and/or a phenotypic change in a cell or animal.

### Target Nucleic Acids

In certain embodiments, compounds described herein comprise or consist of an oligonucleotide that is complementary to a target nucleic acid. In certain embodiments, the target nucleic acid is an endogenous RNA molecule. In certain embodiments, the target nucleic acid encodes a protein. In certain such embodiments, the target nucleic acid is an mRNA. In certain embodiments, an oligonucleotide is complementary to both a pre-mRNA and corresponding mRNA but only the mRNA is the target nucleic acid due to an absence of antisense activity upon hybridization to the pre-mRNA. In certain embodiments, an oligonucleotide is complementary to an exon-exon junction of a target mRNA and is not complementary to the corresponding pre-mRNA.

### Compound Isomers

Certain compounds described herein (e.g., modified oligonucleotides) have one or more asymmetric center and thus give rise to enantiomers, diastereomers, and other stereoisomeric configurations that may be defined, in terms of absolute stereochemistry, as (R) or (S), as α or β, such as for sugar anomers, or as (D) or (L), such as for amino acids, etc. Compounds provided herein that are drawn or described as having certain stereoisomeric configurations include only the indicated compounds. Compounds provided herein that are drawn or described with undefined stereochemistry include all such possible isomers, including their stereorandom and optically pure forms. All tautomeric forms of the compounds provided herein are included unless otherwise indicated.

The compounds described herein include variations in which one or more atoms are replaced with a non-radioactive isotope or radioactive isotope of the indicated element. For example, compounds herein that comprise hydrogen atoms encompass all possible deuterium substitutions for each of the ¹H hydrogen atoms. Isotopic substitutions encompassed by the compounds herein include but are not limited to: ²H or ³H in place of ¹H, ¹³C or ¹⁴C in place of ¹²C, ¹⁵N in place of ¹⁴N, ¹⁷O or ¹⁸O in place of ¹⁶O, and ³³S, ³⁴S, ³⁵S, or ³⁶S in place of ³²S. In certain embodiments, non-radioactive isotopic substitutions may impart new properties on the oligomeric compound that are beneficial for use as a therapeutic or research tool. In certain embodiments, radioactive isotopic substitutions may make the compound suitable for research or diagnostic purposes such as imaging.

### EXAMPLES

### Non-Limiting Disclosure and Incorporation by Reference

Although the sequence listing accompanying this filing identifies each sequence as either “RNA” or “DNA” as required, in reality, those sequences may be modified with any combination of chemical modifications. One of skill in the art will readily appreciate that such designation as “RNA” or “DNA” to describe modified oligonucleotides is, in certain instances, arbitrary. For example, an oligonucleotide comprising a nucleoside comprising a 2′-OH sugar moiety and a thymine nucleobase could be described as a DNA having an RNA sugar, or as an RNA having a DNA nucleobase.

Accordingly, nucleic acid sequences provided herein, including, but not limited to those in the sequence listing, are intended to encompass nucleic acids containing any combination of unmodified or modified RNA and/or DNA, including, but not limited to such nucleic acids having modified nucleobases. By way of further example and without limitation, an oligonucleotide having the nucleobase sequence “ATCGATCG” encompasses any oligonucleotides having such nucleobase sequence, whether modified or unmodified, including, but not limited to, such compounds comprising RNA bases, such as those having sequence “AUCGAUCG” and those having some DNA bases and some RNA bases such as “AUCGATCG” and compounds having other modified nucleobases, such as “ATmCGAUCG,” wherein ^{m}C indicates a cytosine base comprising a methyl group at the 5-position.

While certain compounds, compositions and methods described herein have been described with specificity in accordance with certain embodiments, the following examples serve only to illustrate the compounds described herein and are not intended to limit the same. Each of the references recited in the present application is incorporated herein by reference in its entirety.

### Example 1

### Effects of Uniformly Modified Oligonucleotides Complementary to NCL mRNA

Uniformly modified oligonucleotides complementary to human Nucleolin (NCL) mRNA were designed and tested for their effects on NCL mRNA in vitro. Cultured HeLa cells were transfected with 120 nM of a modified oligonucleotide or no modified oligonucleotide for untreated controls. After approximately 24 hours, RNA was isolated from the cells and NCL mRNA levels were measured by qRT-PCR. A human primer probe set was used to measure mRNA levels. NCL mRNA levels were normalized to total RNA levels as measured by Ribogreen. Results are presented in the table below as normalized NCL mRNA levels, relative to untreated control cells.

The modified oligonucleotides in the table below are each 20 linked nucleosides in length, wherein each internucleoside linkage is a phosphorothioate linkage, and each nucleoside comprises a 2′-MOE modified sugar moiety. The modified oligonucleotides are 100% complementary to the human NCL nucleic acid sequence of GenBank Number NM_005381.2 (designated herein as SEQ ID NO; 1). All of the cytosines are 5-methyl cytosines. “Start Site” indicates the 5′-most nucleoside of the NCL mRNA to which the oligonucleotide is complementary. “Stop Site” indicates the 3′-most nucleoside of the NCL mRNA to which the oligonucleotide is complementary. As shown in the table below, some uniformly modified oligonucleotides complementary to NCL mRNA reduced the amount of NCL mRNA that was present in vitro. The most potent oligonucleotides were complementary to target sites that were closer to the 3′-end of the mRNA coding region than the 5′-end of the mRNA coding region.

TABLE 1
NCL mRNA levels

| | Start | Stop | | | |
|---|---|---|---|---|---|
| | Site on | Site on | | | |
| Compound | SEQ ID | SEQ ID | | NCL mRNA | SEQ |
| Number | NO: 1 | NO: 1 | Sequence (5′ to 3′) | (% control) | ID NO |
| 1199558 | 31 | 50 | CGGAGCACGTACACCCGAAG | 68 | 3 |
| 1199559 | 56 | 75 | TGGCGGCCGCGGGTGCTGAA | 60 | 4 |
| 1199560 | 88 | 107 | AGATGAGTCCAGAAGAAGCC | 82 | 5 |
| 1199561 | 107 | 126 | TGAAGCGGACAAGTGGCGCA | 48 | 6 |
| 1199563 | 190 | 209 | TCCTTTGGAGGAGGAGCCAT | 136 | 8 |
| 1199564 | 216 | 235 | CCTCATCTTCACTATCTTCT | 91 | 9 |
| 1199565 | 238 | 257 | TCTTCTTCATCTTCTGACAT | 86 | 10 |
| 1199566 | 260 | 279 | GACCTCTTCTCCACTGCTAT | 104 | 11 |
| 1199567 | 282 | 301 | TGCCTTTCTTCTGAGGTATG | 100 | 12 |
| 1199568 | 304 | 323 | GCTGAGGTTGCAGCAGCCTT | 37 | 13 |
| 1199569 | 352 | 371 | GGTGTGGCAACTGCAACCTT | 106 | 14 |
| 1199570 | 375 | 394 | GAGTGACAGCTGCTTTCTTG | 61 | 15 |
| 1199571 | 414 | 433 | CTGTCTTCTTGGCAGGTGTT | 112 | 16 |
| 1199572 | 436 | 455 | GTAACTGCTTTGGCTGGTGT | 108 | 17 |
| 1199573 | 458 | 477 | GGCTCCCTTCTTGCCAGGTG | 124 | 18 |
| 1199574 | 481 | 500 | GCTACCAATGCTTTGCCTGG | 63 | 19 |
| 1199575 | 503 | 522 | AGCACCCTTCTTACCAGGAG | 96 | 20 |
| 1199576 | 539 | 558 | ATTCTTGCCATTCTTTGCCC | 108 | 21 |
| 1199577 | 560 | 579 | ATCACTGTCTTCCTTCTTGG | 133 | 22 |
| 1199578 | 582 | 601 | CACTGTCATCATCCTCCTCT | 70 | 23 |
| 1199579 | 623 | 642 | TTCATCCTCATCCTCGTCCT | 97 | 24 |
| 1199580 | 643 | 662 | GCTGCTGGTTCAATTTCATC | 107 | 25 |
| 1199581 | 664 | 683 | GCAGCAGCTGCTGCTTTCAT | 86 | 26 |
| 1199582 | 702 | 721 | CTTCGTCATCCTCATCGTCC | 89 | 27 |
| 1107612 | 722 | 741 | GTCATCGTCATCCTCATCAT | 79 | 28 |
| 1199583 | 744 | 763 | CTTCAGAGTCATCTTCCTCA | 89 | 29 |
| 1199584 | 765 | 784 | GTGTAGTCTCCATAGCTTCT | 110 | 30 |
| 1199585 | 787 | 806 | GCAGCTTTCTTTCCTTTGGC | 92 | 31 |
| 1199586 | 809 | 828 | GGCTTTCACAGGAACAACTT | 85 | 32 |
| 1199587 | 829 | 848 | TCATCCTCAGCCACGTTCTT | 117 | 33 |
| 1107253 | 870 | 889 | CGTCGTCGTCATCCTCGTCC | 110 | 34 |
| 1199588 | 891 | 910 | CATCATCTTCATCATCTTCG | 105 | 35 |
| 1199589 | 912 | 931 | CCTCCTCATCATCTTCATCA | 83 | 36 |
| 1199590 | 934 | 953 | TCTTCCTCCTCCTCTTCTTC | 91 | 37 |
| 1199591 | 955 | 974 | CCAGGTGCTTCTTTGACAGG | 125 | 38 |
| 1199592 | 976 | 995 | GCCATTTCCTTCTTTCGTTT | 115 | 39 |
| 1199593 | 998 | 1017 | TTCAGGAGCTGCTTTCTGTT | 49 | 40 |
| 1199594 | 1021 | 1040 | CCTTCCACTTTCTGTTTCTT | 93 | 41 |
| 1199595 | 1043 | 1062 | GAAAGCCGTAGTCGGTTCTG | 64 | 42 |
| 1199596 | 1065 | 1084 | TTAGGTTTCCAACAAAGAGA | 83 | 43 |
| 1199597 | 1087 | 1106 | TCAGGAGCAGATTTGTTAAA | 121 | 44 |
| 1199598 | 1149 | 1168 | TTCTGACATCCACAACAGCA | 116 | 45 |
| 1199599 | 1191 | 1210 | CAGATTCAAAATCCACATAA | 101 | 46 |
| 1199600 | 1212 | 1231 | ACGCTTTCTCCAGGTCTTCA | 28 | 47 |
| 1199601 | 1234 | 1253 | ACTTTCAAACCAGTGAGTTC | 109 | 48 |
| 1199602 | 1256 | 1275 | TAGTTTAATTTCATTGCCAA | 116 | 49 |
| 1199603 | 1278 | 1297 | TGTCTTTTCCTTTTGGTTTC | 114 | 50 |
| 1199604 | 1299 | 1318 | TCGCATCTCGCTCTTTCTTA | 113 | 51 |
| 1199605 | 1335 | 1354 | TGACTTTGTAAGGGAGATTT | 103 | 52 |
| 1199606 | 1357 | 1376 | ACTTCTTTCAATTCATCCTG | 59 | 53 |
| 1199607 | 1379 | 1398 | GATCTCCGCAGCATCTTCAA | 12 | 54 |
| 1199608 | 1405 | 1424 | CTTTTCCCATCCTTGCTGAC | 102 | 55 |
| 1199609 | 1454 | 1473 | TTTCTCTGCATCAGCTTCTG | 45 | 56 |
| 1199610 | 1476 | 1495 | TTCCCTGCTTTTCTTCAAAG | 67 | 57 |
| 1199611 | 1498 | 1517 | ATAGATCGCCCATCGATCTC | 87 | 58 |
| 1199612 | 1520 | 1539 | CTCTCCAGTATAGTACAGGG | 93 | 59 |
| 1199613 | 1540 | 1559 | TAGTCTTGATTTTGACCTTT | 67 | 60 |
| 1199614 | 1562 | 1581 | AGTGCTATTCTTTCCACCTC | 143 | 61 |
| 1199615 | 1599 | 1618 | GGTTGCTTAAAACCAGAGTT | 101 | 62 |
| 1199616 | 1619 | 1638 | TTCTGTTGCACTGTAGGAGA | 14 | 63 |
| 1199617 | 1640 | 1659 | AAATACTTCCTGAAGAGTTT | 71 | 64 |
| 1199618 | 1660 | 1679 | TTGATAAAAGTTGCTTTCTC | 86 | 65 |
| 1199619 | 1698 | 1717 | CATACCCTTTAGATTTGCCA | 35 | 66 |
| 1199620 | 1720 | 1739 | AATGAAGCAAACTCTATAAA | 95 | 67 |
| 1199621 | 1739 | 1758 | AGCTTCTTTAGCGTCTTCGA | 76 | 68 |
| 1199622 | 1761 | 1780 | CCCTTTTATTACAGGAATTT | 135 | 69 |
| 1199623 | 1782 | 1801 | TGATTGCTCTGCCCTCAATT | 92 | 70 |
| 1199624 | 1803 | 1822 | TGGGTCCTTGCAACTCCAGC | 25 | 71 |
| 1199625 | 1823 | 1842 | TCTGGCATTAGGTGATCCCC | 96 | 72 |
| 1199626 | 1845 | 1864 | ACAGAGTTTTGGATGGCTGG | 35 | 73 |
| 1199627 | 1867 | 1886 | TCCTCAGACAGGCCTTTGAC | 22 | 74 |
| 1199628 | 1889 | 1908 | CTTTAATGTCTCTTCAGTGG | 19 | 75 |
| 1199629 | 1911 | 1930 | GAACGGAGCCGTCAAATGAC | 84 | 76 |
| 1199630 | 1933 | 1952 | CGGTCAGTAACTATCCTTGC | 79 | 77 |
| 1199631 | 2261 | 2280 | CAGAAGCTATTCAAACTTCG | 90 | 78 |
| 1199632 | 2326 | 2345 | TTGATCAGGTAACAGTAAAA | 119 | 79 |
| 1199633 | 2361 | 2380 | ATACTGTCTTGGAATGTCCT | 127 | 80 |
| 1199634 | 2387 | 2406 | GATTTCCAAGGAGACCACAG | 104 | 81 |
| 1107254 | 2420 | 2439 | ACACGGTATTGCCCTTGAAA | 163 | 82 |

### Example 2

### Effects of Uniformly Modified Oligonucleotides Complementary to La mRNA

Uniformly modified oligonucleotides complementary to mRNA transcribed from the human SSB gene, which encodes the La protein, were designed and tested for their effects on La mRNA in vitro. Cultured HeLa cells were transfected with a modified oligonucleotide or no modified oligonucleotide for untreated controls. After approximately 24 hours, RNA was isolated from the cells and La mRNA levels were measured by qRT-PCR. A human primer probe set was used to measure mRNA levels. La mRNA levels were normalized to total RNA levels as measured by Ribogreen. Results are presented in the table below as normalized La mRNA levels, relative to untreated control cells. The modified oligonucleotides in the table below are each 20 linked nucleosides in length, wherein each internucleoside linkage is a phosphorothioate linkage, and each nucleoside comprises a 2′-MOE modified sugar moiety. The modified oligonucleotides are 100% complementary to the human La nucleic acid sequence of GenBank Number NM_003142.4 (designated herein as SEQ ID NO; 2). All of the cytosines are 5-methyl cytosines. “Start Site” indicates the 5′-most nucleoside of the La mRNA to which the oligonucleotide is complementary. “Stop Site” indicates the 3′-most nucleoside of the La mRNA to which the oligonucleotide is complementary. As shown in the table below, some uniformly modified oligonucleotides complementary to La mRNA reduced the amount of La mRNA that was present in vitro. The most potent oligonucleotides were complementary to target sites that were closer to the 3′-end of the mRNA coding region than the 5′-end of the mRNA coding region.

TABLE 2
La mRNA levels

| | Start | Stop | | | |
|---|---|---|---|---|---|
| | Site on | Site on | | | |
| Compound | SEQ ID | SEQ ID | | La mRNA | SEQ |
| Number | NO: 2 | NO: 2 | Sequence (5′ to 3′) | (% control) | ID NO |
| 1199813 | 327 | 346 | ACCTGTTGAATTTTATCATT | 61 | 83 |
| 1199814 | 337 | 356 | AGACGGTTCAACCTGTTGAA | 42 | 84 |
| 1199815 | 347 | 366 | GTCTGTTGTTAGACGGTTCA | 97 | 85 |
| 1199816 | 357 | 376 | TTACATTAAAGTCTGTTGTT | 88 | 86 |
| 1199817 | 367 | 386 | GCTTCCACAATTACATTAAA | 84 | 87 |
| 1199818 | 377 | 396 | TTTGCTCAATGCTTCCACAA | 107 | 88 |
| 1199819 | 387 | 406 | CTGCCTTGGATTTGCTCAAT | 92 | 89 |
| 1199820 | 397 | 416 | TCCATGAGTTCTGCCTTGGA | 87 | 90 |
| 1199821 | 407 | 426 | TTCACTGATTTCCATGAGTT | 96 | 91 |
| 1199822 | 417 | 436 | TAGTTTTATCTTCACTGATT | 70 | 92 |
| 1199823 | 427 | 446 | CTTCTGATTTTAGTTTTATC | 85 | 93 |
| 1199824 | 437 | 456 | GCTTGGAGACCTTCTGATTT | 85 | 94 |
| 1199825 | 447 | 466 | GTAGGGGTTTGCTTGGAGAC | 60 | 95 |
| 1199826 | 457 | 476 | GTCACTTCAGGTAGGGGTTT | 82 | 96 |
| 1199827 | 467 | 486 | ATACTCATCAGTCACTTCAG | 75 | 97 |
| 1199828 | 505 | 524 | CCTTTAATATAAACAGATCT | 73 | 98 |
| 1199829 | 568 | 587 | AGTACTTGACCTTTATCTTC | 80 | 99 |
| 1199830 | 618 | 637 | AAATTGATCCCTTAAATGCT | 78 | 100 |
| 1199831 | 729 | 748 | TTTTGGCAAAGTAATCGTCC | 57 | 101 |
| 1199832 | 774 | 793 | CTCTTAATTTAGCTTCCACT | 59 | 102 |
| 1199833 | 822 | 841 | TTTCAGCATCTTCTTCTAAC | 67 | 103 |
| 1199834 | 1310 | 1329 | TGCTCTTTTCACAGGTCCAG | 113 | 104 |
| 1199835 | 1372 | 1391 | CCAGCACCATTTTCTGTTTT | 81 | 105 |
| 1199836 | 1424 | 1443 | TTTAAAACCTATTTAAAATG | 89 | 106 |
| 1199837 | 1441 | 1460 | CCCGCAAACAAAAGTCGTTT | 92 | 107 |
| 1199838 | 1478 | 1497 | ATTGAAGTGGACCTAATTCG | 72 | 108 |
| 1199839 | 1542 | 1561 | CTCATTTGCATAACAAAAAG | 87 | 109 |
| 1199840 | 1638 | 1657 | ATTCTCATATTACAAAGGCA | 89 | 110 |
| 1199841 | 327 | 346 | ACCTGTTGAATTTTATCATT | 103 | 83 |
| 1199842 | 337 | 356 | AGACGGTTCAACCTGTTGAA | 95 | 84 |
| 1199843 | 347 | 366 | GTCTGTTGTTAGACGGTTCA | 74 | 85 |
| 1199844 | 357 | 376 | TTACATTAAAGTCTGTTGTT | 58 | 86 |
| 1199845 | 367 | 386 | GCTTCCACAATTACATTAAA | 76 | 87 |
| 1199846 | 377 | 396 | TTTGCTCAATGCTTCCACAA | 67 | 88 |
| 1199847 | 387 | 406 | CTGCCTTGGATTTGCTCAAT | 14 | 89 |
| 1199848 | 397 | 416 | TCCATGAGTTCTGCCTTGGA | 19 | 90 |
| 1115471 | 407 | 426 | TTCACTGATTTCCATGAGTT | 2 | 91 |
| 1199849 | 417 | 436 | TAGTTTTATCTTCACTGATT | 30 | 92 |
| 1199850 | 427 | 446 | CTTCTGATTTTAGTTTTATC | 58 | 93 |
| 1199851 | 437 | 456 | GCTTGGAGACCTTCTGATTT | 17 | 94 |
| 1199852 | 447 | 466 | GTAGGGGTTTGCTTGGAGAC | 116 | 95 |
| 1199853 | 457 | 476 | GTCACTTCAGGTAGGGGTTT | 102 | 96 |
| 1115473 | 467 | 486 | ATACTCATCAGTCACTTCAG | 99 | 97 |
| 1199854 | 505 | 524 | CCTTTAATATAAACAGATCT | 88 | 98 |
| 1199855 | 568 | 587 | AGTACTTGACCTTTATCTTC | 91 | 99 |
| 1199856 | 618 | 637 | AAATTGATCCCTTAAATGCT | 97 | 100 |

### Example 3

### Translation dependence of reduction of mRNA by uniformly modified oligonucleotides

Cultured HeLa cells were transfected with 100 nM of a modified oligonucleotide described in Example 1 or no modified oligonucleotide for untreated controls. After 2 hours, 50 μg/mL of cycloheximide (CHX) or control treatment was added to the cells. At various time points after the CHX addition, RNA was isolated from the cells and NCL mRNA levels were measured by qRT-PCR as described in Example 1. As shown in the table below, the reduction of NCL mRNA by uniformly modified oligonucleotide complementary to NCL mRNA was time dependent and was abolished by CHX treatment, indicating that the reduction was dependent on translation.

TABLE 3
NCL mRNA levels

| | NCL mRNA (% control) | | |
|---|---|---|---|
| Time | Compound No. 1199600 | Compound No. 1199600 + CHX | CHX |
| 0 | 100 | 100 | 100 |
| 2 | 81 | 113 | 132 |
| 4 | 67 | 132 | 115 |
| 6 | 54 | 120 | 96 |
| 8 | 39 | 79 | 101 |
| 10 | 30 | 85 | 71 |
| | Compound No. 1199616 | Compound No. 1199616 + CHX | CHX |
| 0 | 100 | 100 | 100 |
| 2 | 71 | 121 | 111 |
| 4 | 49 | 112 | 77 |
| 6 | 33 | 107 | 99 |
| 8 | 23 | 78 | 74 |
| 10 | 22 | 66 | 45 |
| | Compound No. 1199628 | Compound No. 1199628 + CHX | CHX |
| 0 | 100 | 100 | 100 |
| 2 | 59 | 104 | 95 |
| 4 | 54 | 105 | 100 |
| 6 | 40 | 72 | 95 |
| 8 | 23 | 66 | 68 |
| 10 | 18 | 74 | 55 |

### Example 4

### Effects of Modulating No-Go Decay and Nonsense Mediated Decay on the Reduction of mRNA by Uniformly Modified Oligonucleotides

The no-go decay (NGD) and nonsense mediated decay (NMD) pathways were modulated in order to test their effects on inhibition of NCL mRNA by uniformly modified oligonucleotides. Cells were treated with siRNA targeting HBS1L and PELO to modulate the NGD pathway, or with siRNA targeting UPF1 and SMG6 to modulate the NMD pathway, or with luciferase siRNA as a control. The cells were then transfected with a modified oligonucleotide described in Example 1 or 2, or with a uniformly modified oligonucleotide complementary to ACP1 mRNA, or with no oligonucleotide as untreated control. The nucleobase sequences of the uniformly 2′MOE modified ACP1 ASOs are: ACCGTCTCAAAGTCAGAGTC for Compound No.

1217939 (SEQ ID NO: 119) and CTGCTGGTACACCGTCTCAA for Compound No. 1217940 (SEQ ID NO: 120).

After treatment with modified oligonucleotide, RNA was isolated from the cells and NCL or La mRNA levels were measured by qRT-PCR as described in Example 1 or 2, respectively. As shown in Table 4 below, some of the uniformly modified oligonucleotides inhibited their respective target mRNAs via no-go decay; for example, Compound Numbers 1199568, 1199595, 1199600, 1199616, 1199844, and 1199851. As shown in Table 5 below, some of the uniformly modified oligonucleotides inhibited their respective target mRNAs via nonsense mediated decay; for example, Compound Numbers 1199616, 1199626, and 1199628. Thus, some oligonucleotides inhibited their respective targets via one or both mechanisms tested.

TABLE 4
Effects of NGD modulation on target mRNA levels

| | NCL mRNA (% control) | |
|---|---|---|
| Compound Concentration | Compound No. 1199568 + | Compound No. 1199568 + |
| (nM) | Luc. siRNA | HBS1L & PELO siRNA |
| 0 | 100 | 100 |
| 7.5 | 101 | 94 |
| 15.0 | 98 | 104 |
| 30.0 | 86 | 103 |
| 60.0 | 77 | 91 |
| 120.0 | 45 | 75 |
| | Compound No. 1199574 + | Compound No. 1199574 + |
| | Luc. siRNA | HBS1L & PELO siRNA |
| 0 | 100 | 100 |
| 7.5 | 109 | 94 |
| 15.0 | 95 | 92 |
| 30.0 | 98 | 104 |
| 60.0 | 92 | 96 |
| 120.0 | 53 | 78 |
| | Compound No. 1199595 + | Compound No. 1199595 + |
| | Luc. siRNA | HBS1L & PELO siRNA |
| 0 | 100 | 100 |
| 7.5 | 101 | 103 |
| 15.0 | 104 | 116 |
| 30.0 | 101 | 107 |
| 60.0 | 77 | 110 |
| 120.0 | 47 | 95 |
| | Compound No. 1199600 + | Compound No. 1199600 + |
| | Luc. siRNA | HBS1L & PELO siRNA |
| 0 | 100 | 100 |
| 7.5 | 89 | 87 |
| 15.0 | 81 | 90 |
| 30.0 | 59 | 80 |
| 60.0 | 22 | 74 |
| 120.0 | 6 | 50 |
| | Compound No. 1199616 + | Compound No. 1199616 + |
| | Luc. siRNA | HBS1L & PELO siRNA |
| 0 | 100 | 100 |
| 7.5 | 88 | 102 |
| 15.0 | 74 | 105 |
| 30.0 | 46 | 90 |
| 60.0 | 26 | 69 |
| 120.0 | 12 | 54 |
| | La mRNA (% control) | |
| | Compound No. 1199844 + | Compound No. 1199844 + |
| | Luc. siRNA | HBS1L & PELO siRNA |
| 0 | 100 | 100 |
| 7.5 | 104 | 94 |
| 15.0 | 101 | 102 |
| 30.0 | 100 | 98 |
| 60.0 | 91 | 95 |
| 120.0 | 64 | 97 |
| | Compound No. 1199850 + | Compound No. 1199850 + |
| | Luc. siRNA | HBS1L & PELO siRNA |
| 0 | 100 | 100 |
| 7.5 | 26 | 29 |
| 15.0 | 15 | 21 |
| 30.0 | 12 | 16 |
| 60.0 | 7 | 9 |
| 120.0 | 3 | 8 |
| | Compound No. 1199851 + | Compound No. 1199851 + |
| | Luc. siRNA | HBS1L & PELO siRNA |
| 0 | 100 | 100 |
| 7.5 | 92 | 109 |
| 15.0 | 75 | 105 |
| 30.0 | 48 | 102 |
| 60.0 | 30 | 84 |
| 120.0 | 11 | 48 |

TABLE 5
Effects of NMD modulation on target mRNA levels

| | NCL mRNA (% control) | |
|---|---|---|
| Compound Concentration | Compound No. 1199595 + | Compound No. 1199595 + |
| (nM) | Luc. siRNA | UPF1 & SMG6 siRNA |
| 0 | 100 | 100 |
| 7.5 | 93 | 100 |
| 15.0 | 91 | 91 |
| 30.0 | 81 | 86 |
| 60.0 | 63 | 84 |
| 120.0 | 38 | 48 |
| | Compound No. 1199600 + | Compound No. 1199600 + |
| | Luc. siRNA | UPF1 & SMG6 siRNA |
| 0 | 100 | 100 |
| 7.5 | 78 | 83 |
| 15.0 | 63 | 84 |
| 30.0 | 34 | 59 |
| 60.0 | 15 | 27 |
| 120.0 | 9 | 19 |
| | Compound No. 1199607 + | Compound No. 1199607 + |
| | Luc. siRNA | UPF1 & SMG6 siRNA |
| 0 | 100 | 100 |
| 7.5 | 87 | 84 |
| 15.0 | 65 | 64 |
| 30.0 | 38 | 47 |
| 60.0 | 14 | 29 |
| 120.0 | 14 | 36 |
| | Compound No. 1199616 + | Compound No. 1199616 + |
| | Luc. siRNA | UPF1 & SMG6 siRNA |
| 0 | 100 | 100 |
| 7.5 | 72 | 101 |
| 15.0 | 55 | 71 |
| 30.0 | 33 | 52 |
| 60.0 | 13 | 39 |
| 120.0 | 13 | 33 |
| | Compound No. 1199626 + | Compound No. 1199626 + |
| | Luc. siRNA | UPF1 & SMG6 siRNA |
| 0 | 100 | 100 |
| 7.5 | 98 | 107 |
| 15.0 | 84 | 92 |
| 30.0 | 59 | 74 |
| 60.0 | 25 | 60 |
| 120.0 | 20 | 49 |
| | Compound No. 1199628 + | Compound No. 1199628 + |
| | Luc. siRNA | UPF1 & SMG6 siRNA |
| 0 | 100 | 100 |
| 7.5 | 89 | 90 |
| 15.0 | 64 | 80 |
| 30.0 | 38 | 71 |
| 60.0 | 19 | 52 |
| 120.0 | 15 | 47 |
| | ACP1 mRNA levels (% control) | |
| | Compound No. 1217939 + | Compound No. 1217939 + |
| | Luc. siRNA | UPF1 & SMG6 siRNA |
| 0 | 100 | 100 |
| 7.5 | 94 | 76 |
| 15.0 | 89 | 72 |
| 30.0 | 74 | 67 |
| 60.0 | 39 | 45 |
| 120.0 | 16 | 19 |
| | Compound No. 1217940 + | Compound No. 1217940 + |
| | Luc. siRNA | UPF1 & SMG6 siRNA |
| 0 | 100 | 100 |
| 7.5 | 87 | 87 |
| 15.0 | 88 | 74 |
| 30.0 | 65 | 54 |
| 60.0 | 33 | 29 |
| 120.0 | 11 | 7 |

### Example 5

### Effects of Uniformly Modified Oligonucleotides are Dependent on Oligonucleotide Length

Cultured HeLa cells were transfected with a modified oligonucleotide listed in the tables below or no modified oligonucleotide for untreated controls. After approximately 24 hours, RNA was isolated from the cells and NCL mRNA levels were measured by qRT-PCR, as described in Example 1. Results are presented in the table below as normalized La mRNA levels, relative to untreated control cells.

The modified oligonucleotides in the tables below have various lengths, each internucleoside linkage is a phosphorothioate linkage, and each nucleoside comprises a 2′-MOE modified sugar moiety. The modified oligonucleotides are 100% complementary to the human NCL nucleic acid sequence of GenBank Number NM 005381.2 (SEQ ID NO: 1). All of the cytosines are 5-methyl cytosines. “Start Site” indicates the 5′-most nucleoside of the NCL mRNA to which the oligonucleotide is complementary. “Stop Site” indicates the 3′-most nucleoside of the NCL mRNA to which the oligonucleotide is complementary. As shown in the tables below, the oligonucleotides 16 nucleosides in length inhibited the target mRNA more poorly than the longer oligonucleotides tested. Thus, mRNA inhibition by the uniformly modified oligonucleotides is dependent on the length of the oligonucleotide.

TABLE 6
Modified Oligonucleotide Sequences

| | Start | Stop | | | |
|---|---|---|---|---|---|
| | Site on | Site on | | | |
| Compound | SEQ ID | SEQ ID | | | SEQ ID |
| Number | NO: 1 | NO: 1 | Sequence (5′ to 3′) | Length | NO |
| 1288686 | 1212 | 1227 | TTTCTCCAGGTCTTCA | 16 | 111 |
| 1288688 | 1216 | 1231 | ACGCTTTCTCCAGGTC | 16 | 112 |
| 1288685 | 1212 | 1229 | GCTTTCTCCAGGTCTTCA | 18 | 113 |
| 1288687 | 1214 | 1231 | ACGCTTTCTCCAGGTCTT | 18 | 114 |
| 1199600 | 1212 | 1231 | ACGCTTTCTCCAGGTCTTCA | 20 | 47 |
| 1288690 | 1619 | 1634 | GTTGCACTGTAGGAGA | 16 | 115 |
| 1288692 | 1623 | 1638 | TTCTGTTGCACTGTAG | 16 | 116 |
| 1288689 | 1619 | 1636 | CTGTTGCACTGTAGGAGA | 18 | 117 |
| 1288691 | 1621 | 1638 | TTCTGTTGCACTGTAGGA | 18 | 118 |
| 1199616 | 1619 | 1638 | TTCTGTTGCACTGTAGGAGA | 20 | 63 |

TABLE 7
NCL mRNA levels

| | NCL mRNA (% control) | | | | |
|---|---|---|---|---|---|
| Compound Concentration (nM) | 1288686 (16-mer) | 1288688 (16-mer) | 1288685 (18-mer) | 1288687 (18-mer) | 1199600 (20-mer) |
| 0 | 100 | 100 | 100 | 100 | 100 |
| 7.5 | 94 | 97 | 98 | 95 | 81 |
| 15.0 | 98 | 97 | 82 | 87 | 60 |
| 30.0 | 93 | 75 | 65 | 66 | 36 |
| 60.0 | 83 | 45 | 35 | 42 | 17 |
| 120.0 | 65 | 31 | 16 | 21 | 13 |
| | 1288690 (16-mer) | 1288692 (16-mer) | 1288689 (18-mer) | 1288691 (18-mer) | 1199616 (20-mer) |
| 0 | 100 | 100 | 100 | 100 | 100 |
| 7.5 | 100 | 109 | 100 | 83 | 101 |
| 15.0 | 98 | 105 | 76 | 56 | 72 |
| 30.0 | 92 | 91 | 43 | 33 | 38 |
| 60.0 | 91 | 67 | 28 | 22 | 22 |
| 120.0 | 78 | 54 | 25 | 17 | 19 |

What is claimed:

1. An oligomeric compound comprising a modified oligonucleotide consisting of 18-30 linked nucleosides, wherein the modified oligonucleotide is complementary to a target mRNA, wherein the target mRNA is a mature mRNA, and wherein the modified oligonucleotide is not 100% complementary to a corresponding pre-mRNA of the target mRNA.

2. The oligomeric compound of claim 1, wherein the modified oligonucleotide is less than 90% complementary to a corresponding pre-mRNA of the target mRNA.

3. An oligomeric compound comprising a modified oligonucleotide consisting of 18-30 linked nucleosides, wherein the modified oligonucleotide is complementary to a target mRNA, wherein the target mRNA is a mature mRNA, and wherein the modified oligonucleotide is at least 90% complementary to a region within the 3′ half of the coding region of the target mRNA, as measured over the entire length of the modified oligonucleotide.

4. The oligomeric compound of claim 3, wherein the modified oligonucleotide is 100% complementary to a region within the 3′ half of the coding region of the target mRNA, as measured over the entire length of the modified oligonucleotide.

5. The oligomeric compound of claim 1 or 2, wherein the modified oligonucleotide is at least 90% complementary to a region within the 3′ half of the coding region of the target mRNA, as measured over the entire length of the modified oligonucleotide.

6. The oligomeric compound of claim 5, wherein the modified oligonucleotide is 100% complementary to a region within the 3′ half of the coding region of the target mRNA, as measured over the entire length of the modified oligonucleotide.

7. An oligomeric compound comprising a modified oligonucleotide consisting of 18-30 linked nucleosides, wherein the modified oligonucleotide is complementary to a target mRNA, wherein the target mRNA is a mature mRNA, and wherein each nucleoside of the modified oligonucleotide comprises a modified sugar moiety.

8. The oligomeric compound of any of claims 1-6, wherein each nucleoside of the modified oligonucleotide comprises a modified sugar moiety.

9. An oligomeric compound comprising a modified oligonucleotide consisting of 18-30 linked nucleosides, wherein the modified oligonucleotide is complementary to a target mRNA, wherein the target mRNA is a mature mRNA, and wherein the oligomeric compound does not alter splicing of a corresponding pre-mRNA of the target mRNA.

10. The oligomeric compound of any of claims 1-8, wherein the oligomeric compound does not alter splicing of a corresponding pre-mRNA of the target mRNA.

11. An oligomeric compound comprising a modified oligonucleotide consisting of 18-30 linked nucleosides, wherein the modified oligonucleotide is complementary to a target mRNA, wherein the target mRNA is a mature mRNA, and wherein the oligomeric compound induces degradation of the target mRNA.

12. The oligomeric compound of claim 11, wherein the degradation of the target mRNA occurs via no-go decay.

13. The oligomeric compound of claim 11 or 12, wherein the degradation of the target mRNA is dependent on HBS 1L or PELO expression or activity.

14. The oligomeric compound of any of claims 1-10, wherein the oligomeric compound induces degradation of the target mRNA.

15. The oligomeric compound of claim 14, wherein the degradation of the target mRNA occurs via no-go decay.

16. The oligomeric compound of claim 14 or 15, wherein the degradation of the target mRNA is dependent on HBS 1L or PELO expression or activity.

17. An oligomeric compound comprising a modified oligonucleotide consisting of 18-30 linked nucleosides, wherein the modified oligonucleotide is complementary to a target mRNA, wherein the target mRNA is a mature mRNA, and wherein the target mRNA does not contain a premature termination codon.

18. The oligomeric compound of any of claims 1-16, wherein the target mRNA does not contain a premature termination codon.

19. The oligomeric compound of any of claims 1-18, wherein the modified oligonucleotide consists of 18-24 linked nucleosides.

20. The oligomeric compound of claim 19, wherein the modified oligonucleotide consists of 18 linked nucleosides.

21. The oligomeric compound of claim 19, wherein the modified oligonucleotide consists of 19 linked nucleosides.

22. The oligomeric compound of claim 19, wherein the modified oligonucleotide consists of 20 linked nucleosides.

23. The oligomeric compound of claim 19, wherein the modified oligonucleotide consists of 21 linked nucleosides.

24. The oligomeric compound of claim 19, wherein the modified oligonucleotide consists of 22 linked nucleosides.

25. The oligomeric compound of claim 19, wherein the modified oligonucleotide consists of 23 linked nucleosides.

26. The oligomeric compound of claim 19, wherein the modified oligonucleotide consists of 24 linked nucleosides.

27. The oligomeric compound of any of claims 1-26, wherein the modified oligonucleotide is not a gapmer.

28. The oligomeric compound of any of claims 1-27, wherein the modified oligonucleotide does not comprise 5 or more contiguous nucleosides that each comprise a 2′-deoxyfuranosyl sugar moiety.

29. The oligomeric compound of any of claims 1-27, wherein the modified oligonucleotide does not comprise 4 or more contiguous nucleosides that each comprise a 2′-deoxyfuranosyl sugar moiety.

30. The oligomeric compound of any of claims 1-29, wherein the modified oligonucleotide does not comprise any 2′-deoxyfuranosyl sugar moieties.

31. The oligomeric compound of any of claims 1-30, wherein the modified oligonucleotide comprises at least ten nucleosides each comprising a 2′-substituted furanosyl sugar moiety.

32. The oligomeric compound of any of claims 1-30, wherein the modified oligonucleotide comprises at least eleven nucleosides each comprising a 2′-substituted furanosyl sugar moiety.

33. The oligomeric compound of any of claims 1-30, wherein the modified oligonucleotide comprises at least twelve nucleosides each comprising a 2′-substituted furanosyl sugar moiety.

34. The oligomeric compound of any of claims 1-30, wherein the modified oligonucleotide comprises at least thirteen nucleosides each comprising a 2′-substituted furanosyl sugar moiety.

35. The oligomeric compound of any of claims 1-30, wherein the modified oligonucleotide comprises at least fourteen nucleosides each comprising a 2′-substituted furanosyl sugar moiety.

36. The oligomeric compound of any of claims 1-30, wherein the modified oligonucleotide comprises at least fifteen nucleosides each comprising a 2′-substituted furanosyl sugar moiety.

37. The oligomeric compound of any of claims 1-30, wherein the modified oligonucleotide comprises at least sixteen nucleosides each comprising a 2′-substituted furanosyl sugar moiety.

38. The oligomeric compound of any of claims 1-30, wherein the modified oligonucleotide comprises at least seventeen nucleosides each comprising a 2′-substituted furanosyl sugar moiety.

39. The oligomeric compound of any of claims 1-30, wherein each nucleoside of the modified oligonucleotide comprises a 2′-substituted furanosyl sugar moiety.

40. The oligomeric compound of claim 39, wherein each 2′-substituted furanosyl sugar moiety is the same.

41. The oligomeric compound of any of claims 31-40, wherein each 2′-substituted furanosyl sugar moiety is selected from a 2′-O-methyl substituted furanosyl sugar moiety, a 2′-MOE substituted furanosyl sugar moiety, and a 2′-F substituted furanosyl sugar moiety.

42. The oligomeric compound of any of claims 31-40, wherein each 2′-substituted sugar moiety is selected from a 2′-0-methyl substituted furanosyl sugar moiety and a 2′-MOE substituted furanosyl sugar moiety.

43. The oligomeric compound of any of claims 31-40, wherein each 2′-substituted sugar moiety is a 2′-O-methyl substituted furanosyl sugar moiety.

44. The oligomeric compound of any of claims 31-40, wherein each 2′-substituted sugar moiety is a 2′-MOE substituted furanosyl sugar moiety.

45. The oligomeric compound of any of claims 1-30, wherein the modified oligonucleotide comprises at least ten nucleosides each comprising a sugar surrogate.

46. The oligomeric compound of any of claims 1-30, wherein each nucleoside of the modified oligonucleotide comprises a sugar surrogate.

47. The oligomeric compound of claim 46, wherein each sugar surrogate is a morpholino.

48. The oligomeric compound of any of claims 11-47, wherein the degradation of the target mRNA is independent of RNase H1 expression or activity.

49. The oligomeric compound of any of claims 11-48, wherein the degradation of the target mRNA is independent of nonsense mediated decay.

50. The oligomeric compound of any of claims 11-49, wherein the degradation of the target mRNA is independent of UPF1 expression or activity.

51. The oligomeric compound of any of claims 11-50, wherein the degradation of the target mRNA is independent of SMG6 expression or activity.

52. The oligomeric compound of any of claims 1-51, wherein the oligomeric compound does not bind to RNase H1.

53. The oligomeric compound of any of claims 1-52, wherein the oligomeric compound does not support RNase H1 cleavage of the target mRNA.

54. The oligomeric compound of any of claims 1-53, wherein the modified oligonucleotide is less than 90% complementary to an exon-exon junction of the target mRNA.

55. The oligomeric compound of any of claims 1-53, wherein the modified oligonucleotide is not 100% complementary to an exon-exon junction of the target mRNA.

56. The oligomeric compound of any of claims 1-55, wherein the modified oligonucleotide is complementary to a portion of the coding region of the target mRNA that is at least 150 nucleotides downstream from the 5′-end of the coding region of the target mRNA.

57. The oligomeric compound of any of claims 1-55, wherein the modified oligonucleotide is complementary to the 3′ most third of the coding region of the target mRNA.

58. The oligomeric compound of any of claims 1-55, wherein the modified oligonucleotide is complementary to the 3′ most quarter of the coding region of the target mRNA.

59. The oligomeric compound of any of claims 1-58, wherein the modified oligonucleotide is at least 80% complementary to the target mRNA.

60. The oligomeric compound of any of claims 1-58, wherein the modified oligonucleotide is at least 85% complementary to the target mRNA.

61. The oligomeric compound of any of claims 1-58, wherein the modified oligonucleotide is at least 90% complementary to the target mRNA.

62. The oligomeric compound of any of claims 1-58, wherein the modified oligonucleotide is at least 95% complementary to the target mRNA.

63. The oligomeric compound of any of claims 1-58, wherein the modified oligonucleotide is 100% complementary to the target mRNA.

64. The oligomeric compound of any of claims 1-63, wherein the modified oligonucleotide comprises at lease one modified internucleoside linkage.

65. The oligomeric compound of claim 64, wherein the at least one modified internucleoside linkage is a phosphorothioate internucleoside linkage.

66. The oligomeric compound of claim 64, wherein each internucleoside linkage of the modified oligonucleotide is a modified internucleoside linkage.

67. The oligomeric compound of claim 64 or 66, wherein each modified internucleoside linkage of the modified oligonucleotide is the same modified internucleoside linkage.

68. The oligomeric compound of claim 67, wherein each modified internucleoside linkage of the modified oligonucleotide is a phosphorothioate internucleoside linkage.

69. The oligomeric compound of any of claims 64-68, wherein each internucleoside linkage of the oligonucleotide is stereorandom.

70. The oligomeric compound of any of claims 64-68, wherein at least one internucleoside linkage of the oligonucleotide is chirally controlled.

71. The oligomeric compound of any of claims 1-70, wherein the compound comprises a conjugate group.

72. The oligomeric compound of claim 71, wherein the conjugate group comprises GalNAc.

73. The oligomeric compound of any of claims 1-70, wherein the oligomeric compound consists of the modified oligonucleotide.

74. A method comprising contacting a cell with an oligomeric compound of any of claims 1-73.

75. The method of claim 74, wherein the target mRNA is degraded.

76. The method of claim 75, wherein the target mRNA is degraded by no-go decay.

77. The method of claim 74 or 75, wherein the target mRNA degradation is dependent of HBS1L or PELO expression of activity.

78. The method of any of claims 74-77, wherein the cell is in an animal.

79. The method of any of claims 74-77, wherein the cell is in a human.

80. A method of treating a disease or disorder, comprising administrating the oligomeric compound of any of claims 1-73 to an individual in need thereof.

81. The method of claim 80, wherein the individual is an animal.

82. The method of claim 80, wherein the individual is a human.

83. The method of any of claims 80-82, wherein the administration is systemic.

84. The method of claim 83, wherein the administration is subcutaneous.

85. The method of any of claims 80-82, wherein the administration is intrathecal.

86. The method of any of claims 80-82, wherein the administration is via inhalation.

87. The oligomeric compound of any of claims 1-73, for use in treating a disease or disorder.
